(19) United States
(12) Patent Application Publication
(10) Pub. No.: US 2024/0428951 A1
(43) Pub. Date: Dec. 26, 2024

(54) METHODS AND SYSTEMS FOR PERSONALIZED IN-SILICO HEMATOPOIESIS AND DISEASE/LEUKEMIA SIMULATION FOR TREATMENT SELECTION AND OPTIMIZATION

(71) Applicants: GEORGIA TECH RESEARCH CORPORATION, Atlanta, GA (US); EMORY UNIVERSITY, Atlanta, GA (US)

(72) Inventors: Nicki PANOSKALTSIS, Atlanta, GA (US); Athanasios MANTALARIS, Atlanta, GA (US); Ana Luz Quiroga CAMPANO, Atlanta, GA (US)

(21) Appl. No.: 18/701,544

(22) PCT Filed: Oct. 14, 2022
(86) PCT No.: PCT/US2022/046722

§ 371 Date: Apr. 15, 2024

Related U.S. Application Data
(60) Provisional application No. 63/256,234, filed on Oct. 15, 2021.

(51) Int. Cl. G16H 50/50; G16H 10/60; G16H 15/00; G16H 20/17

(52) U.S. Cl. CPC G16H 50/50; G16H 10/60; G16H 15/00; G16H 20/17

(57) ABSTRACT

The systems and methods can generate simulations using a stored mechanistic deterministic mathematical model based on patient specific clinical information and therapeutic data (e.g., pharmacokinetics and pharmacodynamics) to predict a patient response (e.g., cell numbers) to a given therapy. The model simulations can be used to (1) predict a leukemia response to a given treatment therapy, (2) predict patient recovery to that given treatment therapy, and/or (3) optimize treatment therapy.

### CROSS-REFERENCE TO RELATED APPLICATIONS

This application claims the benefit of U.S. Provisional Application No. 63/256,234 filed Oct. 15, 2021, which is hereby incorporated by reference herein in its entirety for all purposes.

### BACKGROUND

Leukemia, a cancer of the bone marrow, peripheral blood, and innate immune system, still has a poor prognosis and overall survival despite the development of new treatments, such as intensive chemotherapy combinations and therapeutic agents (including drugs and cells) targeting the products of mutated genes, epigenetic mechanisms, or active surface receptors (e.g., immunotherapies, small molecule inhibitors (SMIs) and hypomethylating agents (HMAs)). Dosing of intensive and non-intensive chemotherapy combinations over the last 50 years has generally been based solely on body surface area, renal/hepatic function, and pharmacokinetics/pharmacodynamics (PK/PD). Consequently, under-and over-dosage can occur in a majority of obese patients due to capping of body weight, and a further 10-40% of patients can have a primary refractory disease to gold-standard first-line treatment. These treatments generally ignore-specific parameters, such as tumor burden, heterogeneity, or cell cycle kinetics, and thus are neither patient-nor-leukemia specific. Therefore, the current dosing can result in an empirical approach that results in poor outcomes and high treatment costs.

More recently, in the last 10 years, the therapeutics targeting the products of mutated genes, epigenetic mechanisms, or active surface receptors, such as immunotherapies, small molecule inhibitors (SMIs), and hypomethylating agents (HMAs), have been approved for use with or without chemotherapy. While these novel targeted therapeutics have resulted in improved remission rates, often in combination with backbone chemotherapy, overall survival remains unchanged, for example, due to leukemic clonal heterogeneity, which is different in every patient, and the dynamic evolution of mutational profiles during treatment causing resistance.

Unfortunately, hematologists and oncologists lack the tools to predict patient responses to a given therapy. Therefore, patients can undergo suboptimal therapies that may be insufficiently effective (under-dosage), toxic (overdosage), or ineffective (resistant disease), and another empirically selected therapeutic regimen must be attempted.

### SUMMARY

Therefore, there is a need for precision oncology medicine systems that can efficiently and accurately predict patient responses to a given therapy, thereby improving healthcare outcomes.

In some embodiments, the systems and methods of the disclosure can generate simulations using a stored mechanistic, deterministic mathematical model based on patient-specific clinical information and therapeutic data (e.g., pharmacokinetics and pharmacodynamics) for a single individual patient to predict a patient response (e.g., cell numbers) to a given therapy for that patient. For example, these multi-module model simulations can (1) predict a leukemia response to a given treatment therapy, (2) predict patient recovery to that given treatment therapy, and/or (3) optimize treatment therapy. Therefore, the systems and methods of the disclosure can improve efficacy and reduce toxicity, thereby improving survival (e.g., by controlling and/or curing the disease) and patient quality of life. The use of mechanistic, deterministic mathematical models for an individual patient beneficially provides an objective measure of that patient's response in which the modeling and analysis can be performed without data from other patients that could introduce bias.

In some embodiments, the disclosed embodiments can be employed to simulate the patient response for a hypothetical patient to evaluate or drive the development of new therapeutics or cell therapy.

In some embodiments, the disclosed embodiments may include computer-implemented systems and methods for simulating one or more treatment responses to different therapeutics or cell therapy for a patient having a disease to determine one or more metrics for the different therapeutics or therapy, e.g., to determine an optimal treatment for a patient having a disease. In some embodiments, a method may include receiving patient and/or simulation information for a patient. The patient information may include normal cell population information and abnormal cell population information. The simulation information may include current or proposed therapeutic or therapy information. The method may then include generating simulations of the progression of each cell cycle of abnormal and normal cell populations in and between phases of each cell cycle in a target organ for a period of time using a stored model (e.g., the mechanistic, deterministic mathematical model), simulation parameter(s) or simulation information, and the patient information to predict one or more metrics at intervals during and at the end of the period of time. In some embodiments, the one or more metrics may include one or more disease metrics, one or more recovery metrics, among others, or any combination thereof.

In some embodiments, a system may include one or more processors; and one or more hardware storage devices having stored thereon computer-executable instructions. The instructions may be executable by the one or more processors to cause the computing system to perform receiving patient information for a patient, the patient information including normal cell population information and abnormal cell population information. The one or more processors may be further configured to cause the computing system to perform generating simulations (e.g., mechanistic simulation) of the progression of cell cycles of abnormal and normal cell populations in and between phases of each cell cycle in a target organ for a period of time using a stored model, simulation parameter(s), and the patient information to predict one or more metrics at intervals during and at an end of the period of time In some embodiments, the one or more metrics may include one or more disease metrics, one or more recovery metrics, among others, or any combination thereof.

In some embodiments, the target organ may be bone marrow and/or peripheral blood. In some embodiments, the disease may be a blood disorder, such as leukemia.

In some embodiments, the one or more disease metrics may include a percentage of diseased cells, a type of diseased cells, minimal/measurable residual disease (MRD), sub-clonal evolution, cytokine/chemokine or other plasma or serum protein levels, among others, or a combination thereof.

In some embodiments, the one or more disease metrics and/or one or more recovery metrics may include (i) an absolute number of normal stem cells, progenitors, precursors, and an absolute number of mature hematopoietic and immune cells, (ii) an absolute number of mature hematopoietic and immune cells in the blood stream, and/or (iii) immune-related adverse event(s) (e.g., cytokine storm), among others, or a combination thereof.

In some embodiments, the receiving step may include treatment information for one or more potential treatment therapies. In some embodiments, the method and/or the one or more processors may be further configured to cause the computing system to further perform determining one or more PK/PD metrics using for each potential treatment therapy using PK/PD parameters specific to each potential treatment therapy. The one or more PK/PD metrics may be employed in the mechanistic simulation to generate the one or more disease metrics and/or the one or more recovery metrics.

In some embodiments, the one or more disease metrics or one or more recovery metrics may include a therapeutic concentration in peripheral blood and/or the target organ or tissue.

In some embodiments, the one or more disease metrics or one or more recovery metrics may include adjustment to the cell therapy protocol defined for the patient.

In some embodiments, the receiving of the treatment information may include receiving only a PD therapeutic effect, for example, inputted by a user. In some embodiments, the generating of the simulations may include simulating a response of the normal and abnormal populations to a treatment therapy having the PD therapeutic effect. In some embodiments, the method and/or the one or more processors may be further configured to cause the computing system to further perform determining one or more novel therapeutic targets using the one or more metrics.

In some embodiments, the generating simulations of the progression of each cell cycle of abnormal and normal cell populations include generating simulations in stages within each phase of each cell cycle.

In some embodiments, the normal cell population information may include cell type, and the abnormal cell population information may include cell type and/or aberrations. In some embodiments, the one or more simulation parameters may include cell cycle time, cellular metabolism, interaction with their microenvironment, and interaction with the microenvironment of the target organ, including cytokine responses, adhesion, kinetics, and/or pharmaceutical sensitivity.

In some embodiments, the one or more disease metrics may include sub-clonal kinetics (e.g., growth rate) and/or evolution (e.g., percentage of each sub-clone within the tumor) based on the cell-type and/or the aberrations of the abnormal cell population.

In some embodiments, for the normal cell population, the progression of and in between the cell cycle may be based on a cell type according to a hemopoietic differentiation tree.

In some embodiments, each simulation of the cell cycle for normal and/or abnormal cell populations within each phase and/or stage may be based on the simulation parameter(s). The parameters for normal and abnormal populations may be based on (i) hematopoietic cell type or (ii) hematopoietic cell type and aberration types, respectively.

In some embodiments, the parameters for normal cell populations may include cell cycling time, recruitment rate, apoptosis rate, sensitivity to therapeutics, among others, or a combination thereof. In some embodiments, the parameters for abnormal cell populations include cell cycle time, and sensitivity to therapeutics, among others, or a combination thereof.

Additional advantages of the disclosure will be set forth in part in the description which follows, and in part will be obvious from the description or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with reference to the following drawings and description. The components in the figures are not necessarily to scale, the emphasis being placed upon illustrating the principles of the disclosure.

FIG. 1A illustrates an example of a system environment for generating multi-module model simulations of cell populations to predict a patient's personalized disease state and/or response to different therapeutic treatment alternatives to determine an optimal treatment according to embodiments.

FIG. 1B illustrates an example of another system environment for generating multi-module model simulations of cell populations to predict a patient's personalized disease state and/or response to cell therapy treatment to determine an optimal treatment according to embodiments.

FIG. 2 is a flow chart illustrating an example of a method of generating multi-module model simulations of cell populations to determine a patient's personalized disease state and/or response to different treatment alternatives and to determine an optimal treatment according to embodiments.

FIG. 3 shows a flow chart illustrating an example of generating PK/PD outputs using a PK/PD module according to embodiments.

FIG. 4 shows a flow chart illustrating an example of using a population simulation engine to generate metrics according to embodiments.

FIG. 5 shows an example of a multi-module model simulation of a cell population of FIG. 4 with cell cycle phases and stages according to embodiments.

FIGS. 6A and 6B show examples of visual representations of dynamic prediction of an absolute number of normal cells in the BM during treatment determined using the method according to embodiments. FIG. 6A shows an example of a patient under 3 cycles of low-dose Cytosine Arabinoside (Ara-C) treatment with one sensitive and one resistant sub-clonal population. FIG. 6B shows an example of a different patient under 4 cycles of intensive Ara-C and Daunorubicin (DNR) treatment with the sensitive disease only.

FIGS. 7A and 7B show examples of visual representations of innate immune cells and apoptotic debris and respective cytokines in the BM during treatment, respectively, determined using the method according to embodiments.

FIGS. 8A and 8B show examples of visual representation of dynamic prediction of an absolute number of total normal cells, and sensitive and resistant sub-clones determined using the method according to embodiments. FIG. 8A shows an example of a patient under 4 cycles of low-dose Ara-C treatment with one sensitive disease clone only. FIG. 8B shows an example of a different patient under 4 cycles of intensive Ara-C and DNR treatment with a dominant initial sensitive sub-clonal population, which is eliminated during treatment, and a small resistant population, with aggressive growth, that overtakes the patient bone marrow producing relapse.

FIGS. 9A and 9B show examples of visual representations of retrospective comparisons of dynamic prediction of the overall percentage of abnormal cells/leukemic blasts (lines) determined using the method according to embodiments and patient clinical assessments (dots). FIG. 9A shows an example of a patient under 2 cycles of low-dose Ara-C treatment that achieved complete remission (<5% leukemic blasts). FIG. 9B shows an example of a different patient treated with 3 cycles of intensive Ara-C and DNR treatment—the 2 initial cycles were successful, and the 3^{rd} cycle was ineffective due to the predicted growth of a resistant and aggressive sub-clone that overtakes the patient BM, causing relapse.

FIGS. 10A and 10B show examples of visual representations of dynamic prediction of peripheral blood recovery determined using the method according to embodiments. FIG. 10A shows an example of a patient under 4 cycles of low-dose Ara-C treatment that achieved complete remission and complete hematological recovery. FIG. 10B shows an example of a different patient under 2 cycles of intensive Ara-C and DNR that achieved complete remission and is achieving complete hematological recovery.

FIGS. 11A and 11B show examples of visual representations of retrospective comparisons of dynamic prediction of absolute neutrophils count (lines) determined using the method according to embodiments and patient clinical assessment of daily neutrophils count during treatment (dots). FIG. 11A shows an example of a patient under 4 cycles of low-dose Ara-C treatment. FIG. 11B shows an example of a different patient under 2 cycles of intensive Ara-C and DNR that achieved complete remission and complete hematological recovery.

FIG. 12 shows a diagram of visual comparisons of dose, cumulative dose, administration method (bolus injection or infusion), and schedule between actual treatment and optimized treatment therapy determined using the method according to embodiments.

FIG. 13 shows a visual comparison of the absolute number and percentage of leukemic blasts between actual treatment and optimized treatment therapy determined using the method according to embodiments.

FIG. 14 shows a visual comparison of an absolute number of neutrophils between actual treatment (dots) and optimized treatment therapy (line) determined using the method according to embodiments.

FIG. 15 is a simplified block diagram of an example of a computing system for implementing certain embodiments disclosed herein.

### DESCRIPTION OF THE EMBODIMENTS

In the following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

The disclosed embodiments relate to systems and methods for generating patient-specific predictive simulations of cell cycles using a multi-module model that predicts disease state with personalized and dynamic responses to different treatment alternatives, FDA-approved for the specified disease or for other indications, or in a clinical trial, or pre-clinical evaluation, and/or without treatment intervention for treatment selection for a disease, such as leukemia. The systems and methods of the disclosure use these models to simulate the patient's disease state over a period of time to determine (predict) one or more disease and/or recovery metrics. By way of example, the disease metrics and/or recovery metrics may include but is not limited to patient bone marrow (e.g., absolute number of normal stem cells, absolute number of progenitors, absolute number of precursors, absolute number of mature hematopoietic cells, and/or absolute number of immune cells) and peripheral blood recovery (e.g., absolute number of mature hematopoietic and absolute number of immune cells in the blood stream), among others, or any combination thereof; the one or more disease metrics or one or more recovery metrics may include but are not limited to one or more physiological PK/PD metrics, such as therapeutic concentration in blood, therapeutic concentration in the target organ, among others, or any combination thereof; disease state (also referred to as “disease”) metrics may include but are not limited to the absolute number of abnormal sub-clones, percentage of abnormal sub-clones, measurable/minimal residual disease (MRD) (e.g., absolute number of sub-clonal leukemic cells in the bone marrow), the amount of disease remaining after treatment as measured by sensitive immunophenotyping, molecular or other more sensitive measurements, clonal evolution (i.e., percentage of each diseased/mutated and normal cell sub-clone in the tumor and normal tissues over time), immune-related adverse events (iRAES) (e.g., the cytokine and immune cell imbalance which corresponds to symptoms related to treatment-related immune-mediated pathology); among others, or a combination thereof; and recovery metrics may include but are not limited to a hematological recovery metric (e.g., when the cell number in BM and PB reaches the normal range). Using these personalized simulations and metrics, the systems and methods therefore can: (1) provide healthcare professionals with predictions of patient response to different treatment alternatives or treatment selection and (2) determine an optimal personalized dose, schedule, and route of delivery of a treatment therapy that can offer better outcomes and toxicity reductions, thereby reducing hospitalization time and risk of complications. This can therefore allow a strategic administration of targeted therapeutics in-line with the personalized clonal dynamics to anticipate and prevent a disease state. Various embodiments are described herein, including systems, methods, devices, modules, models, algorithms, networks, structures, processes, computer-program products, and the like.

In some embodiments, the simulations using the multi-module model can be initialized and generated using patient clinical information (e.g., age, height, weight, cardiac/renal/liver function, and/or comorbidities); disease information (e.g., for leukemia, disease status (e.g., Secondary or De Novo), disease type (e.g., World Health Organization Classification), bone marrow differential, histology, flow cytometry immunophenotyping, cytogenetic, and/or molecular assessments and/or peripheral blood total cell count); treatment information (e.g., therapeutic, dose, administration type (e.g., oral, subcutaneous or intravenous), administration method (e.g., bolus injection or infusion) and/or schedule (e.g., date and timing)); among others; or a combination thereof. By incorporating these data into the model-based simulations, inter-and intra-patient and tumor-response heterogeneity can be considered, thereby resulting in metrics that are highly personalized and accurate.

For example, using the patient and disease information (e.g., fixed patient clinical data and tumor characterization data) acquired at the patient assessment and treatment information (e.g., treatment and regimen specifications for each potential treatment therapy), the simulations using the multi-module model can determine (predictive) disease metrics or recovery metrics for each treatment therapy. By way of example, for leukemia, the simulations of the disclosure can predict normal hematopoietic and leukemic cell numbers in the bone marrow and peripheral blood until the first treatment cycle induces a perturbation, with each administrated dose, and the method can predict its effect on normal blood production and disease state. Throughout the entire course of treatment (without limitation of cycle number or treatment length) and years after treatment, the methods and systems of the disclosure can dynamically determine normal cell recovery, disease state, and/or clonal evolution. The simulations and metrics may be generated for each potential treatment. In this example, the methods and systems of the disclosure can determine metrics that represent a personalized dynamic patient response to each treatment and provide such metrics in a report.

By way of example, the methods and systems of the disclosure can determine one or more metrics that predict efficacy and detailed dynamic hematological, disease (e.g., leukemia), and immunological response to potential therapeutics in preclinical and clinical trials. For example, the methods and systems may simulate the mechanism of action of a new therapeutic and virtual administration of a treatment, such as a novel therapeutic to a real patient, and provide in-silico results to determine (predict) disease metrics or recovery metrics (e.g., number of hematopoietic, leukemic and immune cells in the bone marrow and/or peripheral blood) to assess the disease and/or recovery metrics (e.g., MRD, clonal evolution, immune-related adverse events (iRAEs) and/or hematological recovery).

By way of another example, the methods and systems of the disclosure can determine one or more metrics that predict dynamic hematological recovery. For example, the methods and systems of the disclosure can generate simulations using patient and disease information acquired at patient assessment and treatment administration to determine the disease metrics and/or recovery metrics (e.g., cell count in the BM and PB: the dynamic absolute number of red blood cells, white blood cells and/or lymphocytes). These metrics can be used to determine recovery metric(s) (i.e., when the cell number in BM and PB reach the normal range) that can represent a prediction of when a patient could reach complete hematological recovery.

By way of a further example, the methods and systems of the disclosure can predict the final overall outcome. For example, the methods of the systems of the disclosure can generate the multi-module simulations using patient and disease information acquired at patient assessment and treatment administration to determine an overall outcome (e.g., disease metric(s) at the end of each treatment course). For example, the overall outcome may be complete remission, partial remission, relapse, or resistance.

By way of a further example, the methods and systems of the disclosure can optimize the treatment therapy (e.g., dose, scheduling, duration of injections, and/or administration method). In some examples, the treatment therapy can be constrained by maximum cumulative dose/day, renal or hepatic and cardiac impairment (e.g., actual or at risk) dose-limitations, based on FDA or other treatment-related guidance. For example, the generated simulations can minimize the number of leukemic cells to achieve morphologic complete remission (CR) while maintaining (i) the number of normal cells above hematological toxicity levels to reduce the risk of BM failure and ensure recovery and (ii) the absolute cell count in peripheral blood to avoid cytopenias.

In further examples, the methods and systems of the disclosure can generate dynamic predictive simulations of patient/tumor-personalized hematological, leukemia and immunological response to a treatment using the multi-module model to determine the metrics related to the recovery of normal cells, disease state, and clonal evolution. The multi-module mechanistic and deterministic model of the disclosure can be based on: i) the structure of the hematopoietic differentiation tree, including myeloid-derived immune cells, lymphoid-progenitor cells, among others, ii) the effect of cell-specific therapeutics dependent on cell type, biomarkers and cell cycle stage, iii) interaction between normal and abnormal populations, based on the homeostatic number of each cell type in the hematopoietic tree and the detrimental effect on leukemic cells, and/or iv) cell cycle times and differentiation recruitment rates between cycling and quiescent compartments, which can be variable and depend on the interaction of all cells in the target organ, such as bone marrow.

The systems and methods of the disclosure can provide quantitative disease evolution (progression/regression) with and without treatment over time with graphical visualization and optimal dynamic personalized recovery for each individual patient prospectively in response to the whole landscape of FDA-approved therapeutics or in clinical trials, prior to starting therapy. Using the system and methods of the disclosure, treatments can be therefore more quickly, effectively, and efficiently identified with respect to the goals of care of the physician and the patient's expressed treatment goals, outcome, recovery, and cost.

Treatment (also referred to as “treatment therapy”) refers to any therapeutic treatment including but not limited to a pharmaceutical, cell therapy, including the treatment of patients with stem cell transplants and recovery, immune cells and cytokines, in vivo or ex vivo inhibition/interference methods, or gene therapy approaches, among others, or any combination thereof. Pharmaceutical treatment may include but is not limited to an antibody, cytokine, and/or drug (e.g., one or more compositions). Cell therapy may include but is not limited to the treatment of patients with stem cell transplants and prediction of recovery kinetics, stem cell mobilization protocols, and other relevant treatment protocols for cell therapy and graft vs. host disease. For example, cell therapy may include but is not limited to different types of T cells, NK cells, monocytes/macrophages, different types/subtypes of T cells and NK cells or other cell types, or engineered cell therapies such as CART or CAR-NK cells, etc.

It will be understood that the methods of the systems of the disclosure are not limited to treating leukemia discussed herein and may be used to treat other cancers, diseases, and/or disorders. For example, the other cancers may include but are not limited to acute myeloid leukemia, and other myeloid neoplasms such as myelodysplastic syndrome, myeloproliferative neoplasms, chronic myelomonocytic leukemia, chronic myeloid leukemia, other types of leukemia and hematopoietic neoplasms (e.g., chronic lymphocytic leukemia, acute lymphoblastic leukemia, mast cell disease, multiple myeloma, other plasma cell neoplasms, etc.), clonal hematopoiesis and related conditions, aplastic anemia, among others, or any combination thereof. By way of another example, the systems and methods of the disclosure could also be applied to clonal and sub-clonal disease inherent in other tumors with related imaging modalities for size and volume assessments such as Hodgkin and Non-Hodgkin Lymphomas, or solid tumors of different tissue or organ types.

It will also be understood that the methods and systems of the disclosure are not limited to generating patient-specific predictive simulations of cell cycles using a multi-module model, for example, to determine (predict) one or more disease and/or recovery metrics associated with personalized and dynamic response to different therapeutic treatment alternatives discussed herein.

In some embodiments, the methods and systems of the disclosure can be used to determine potential sub-clonal target(s) that could correspond to therapeutic target(s) for therapeutic discovery and/or development. For example, virtual pharmacodynamic effects (e.g., via an inputted PD therapeutic effect) may be introduced into the model that may target certain sub-clonal populations to simulate the sub-clonal evolution and can be used to define the best sub-clonal target or targets to achieve a complete remission with complete hematological recovery. The sub-clonal population(s) can be segregated by cell-type and aberrations they carry, which define simulation parameter(s) related to the sub-clonal metabolism (e.g., activated metabolic pathways), sub-clonal kinetics (e.g., sub-clonal cell cycle time, growth rate, and/or tumor clonal evolution), and therapeutic-specific sensitivity in the simulation. Therefore, the systems and method of the disclosure can select strategic “sub-clones or cell-type/co-occurrent aberrations” with a specific metabolic, cytokine, gene or protein signature and identify more sophisticated therapeutic targets.

### Example System #1

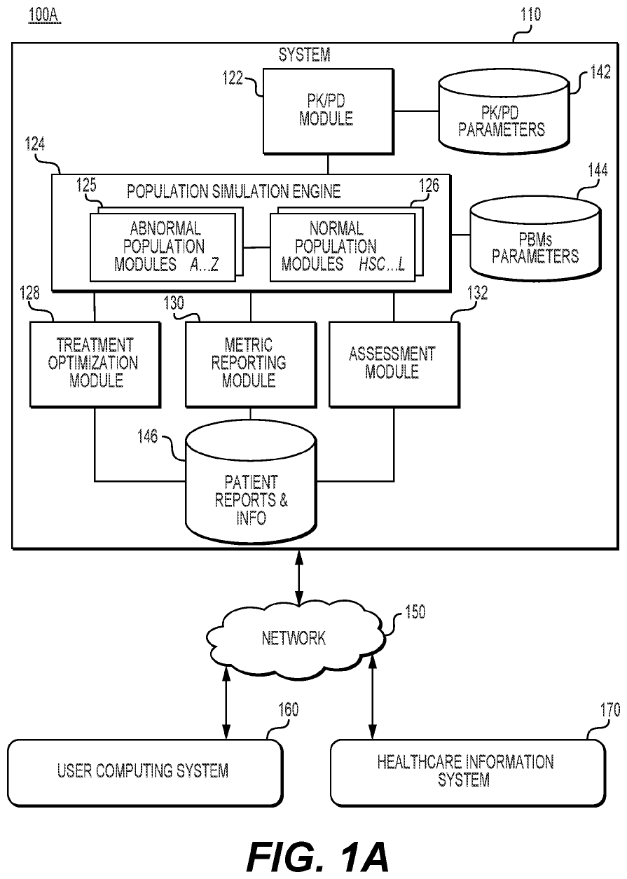

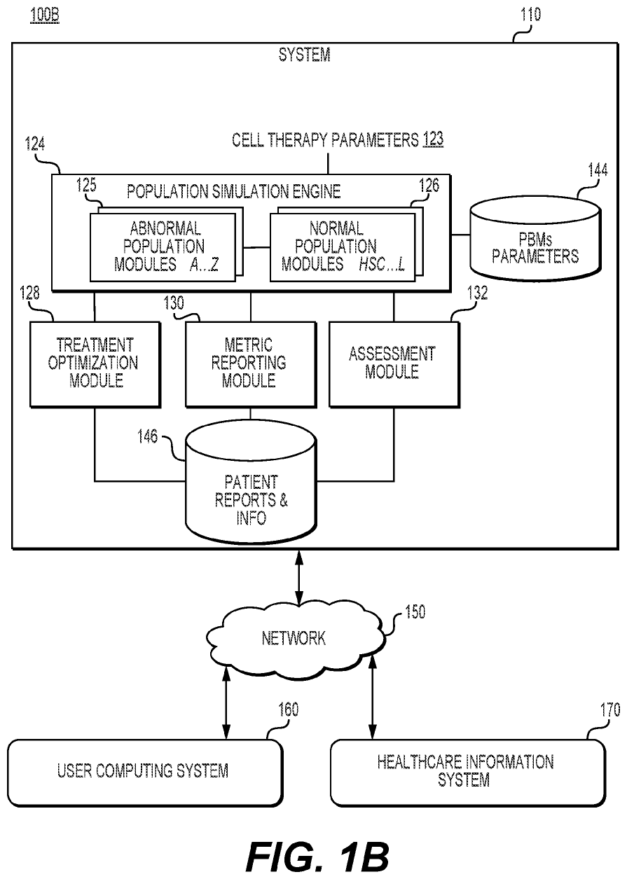

FIG. 1A depicts an example of a system environment 100 (shown as 100A) for generating patient-specific simulations using the mathematic, mechanistic, deterministic, and modular model and related information (e.g., disease and patient with or without treatment information) in a target organ (i) to predict a disease state (e.g., without treatment information) by determining one or metrics, and/or (ii) to predict a patient response to a treatment therapy by determining one or more metrics for FDA-approved-treatment selection, and/or (iii) to determine an optimal treatment therapy based on the metrics according to some embodiments. In some embodiments, using these simulations, the system 100A may also determine patient, treatment, disease, and/or recovery metrics, and generate reports (computerized or otherwise) using the metric(s), among others, or a combination thereof.

In some embodiments, the environment 100A may include a system 110 configured to generate patient-specific model-based simulations for a target organ (e.g., bone marrow (BM)) using the patient information and treatment information for each treatment therapy to predict disease state and/or response to each treatment therapy to determine the one or more metrics, one optimal treatment, among others, or a combination thereof. In some embodiments, the system 110 may include any computing or data processing device consistent with the disclosed embodiments. In some embodiments, the system 110 may incorporate the functionalities associated with a personal computer, a laptop computer, a tablet computer, a notebook computer, a hand-held computer, a personal digital assistant, a portable navigation device, a mobile phone, an embedded device, a smartphone, and/or any additional or alternate computing device/system. The system 110 may transmit and receive data across a communication network 150.

In some embodiments, the system 110 may use patient information and/or treatment information for each treatment therapy inputted by a clinician using a user computing system 160 and/or retrieved from a healthcare information system 170. For example, the healthcare information system 170 may include but is not limited to hospital information systems (HIS), radiology information systems (RIS), clinical information systems (CIS), pathology information systems (PIS), and cardiovascular information systems (CVIS), and storage systems, such as picture archiving and communication systems (PACS), library information systems (LIS), and electronic medical records (EMR), among others, or any combination thereof. Information stored/transmitted can include, for example, patient medication orders, medical histories, imaging data, pathology data (including histology, immunophenotypic, molecular, and/or more sensitive testing techniques), test results, diagnosis information, management information, and/or scheduling information, among others, or a combination thereof.

In some embodiments, the patient information may include but is not limited to patient clinical information, disease characterization information (also referred to as “disease information”), among others, or a combination thereof. For example, the patient clinical information may include but is not limited to a number of normal cells in BM and peripheral blood (PB), determined by BM differential analysis and PB total cell count; patient physiological information (e.g., age, age, height, weight, gender, cardiac/renal/liver function in percentage, among others, or a combination thereof); comorbidity score (e.g., defined by ECOG or Karnosfsky performance score and as impacted by age, physical deconditioning, diabetes, hypertension, other confounding medical illnesses, etc.); among others; or a combination thereof.

By way of further example, for leukemia, the disease characterization information may include but is not limited to the target organ (e.g., BM, disease status (e.g., Secondary, Transformed, or De Novo), leukemia type, BM differential, flow cytometry immunophenotyping assessment results, histology results, cytogenetic results, molecular genetic results, peripheral blood total cell count, the absolute number of each sub-clonal population (e.g., including its mutation profile, cell types and/or location in the target; and/or including organ/spatial single-cell multi-omics assessment when available), among others, or a combination thereof. For example, the disease or type of cancer can define the target organ. In some examples, the tumor characterization information may include results from additional tests, which may depend on the type of cancer. For example, for solid tumors, tumor characterization information may include results from additional tests such as biopsy-specific multi-omics (e.g., genomics/proteomics/immune cell and/or activation profile) assessments and/or imaging of the tumor and vascularization results. In some examples, the results included in the tumor characterization information may be collected from routine oncology diagnostic and standard of care testing performed with the healthcare provider. For example, the calculation of the absolute number of each sub-clonal tumor and normal cell population, including its mutation profile, cell types and location in the target organ, and/or the number of sub-clonal leukemic populations and non-leukemic defined by the aberrations/mutations that they carry may be determined by a target organ tumor biopsy/aspirate or blood analysis (e.g., using Next Generation Sequencing or single-cell multi-omics or similar genomic and/or immunophenotypic technique, among others, or a combination thereof).

In some embodiments, the treatment information for each treatment therapy may include treatment regimen, pharmacological parameters, cell or biologic parameters, among others, or a combination thereof. Each treatment therapy may include one treatment and/or a combination of treatments. For example, for a therapeutic treatment, a treatment may include one therapeutic (e.g., drug), a combination of therapeutics (e.g., drugs), one immunotherapeutic, a cocktail of immunotherapeutics, one cell therapy, a cocktail of cell therapies, a combination of systemic therapies, among others described herein, or a combination thereof. By way of example, for leukemia, treatments may include Cytarabine (ARA-C), Daunorubicin (DNR), or Gemtuzumab Ozogamicin (GO), among others, and/or a combination thereof. For a therapeutic treatment, the treatment regimen may include but is not limited to therapeutic, dose, administration type (oral, subcutaneous, or intravenous), administration method (bolus injection or infusion), and schedule (timing). For example, for treatment simulations, the treatment regimen for a therapeutic treatment therapy may be from the “Package leaflet Information for the user,” FDA-approval packaging, therapeutic company information, hospital clinical protocols, among others, or a combination thereof. The pharmacological parameters may include but are not limited to absorption rate, bioavailability (F), maximum plasma concentration (Cmax), time until Cmax is reached (Tmax), area under the concentration-time curve (AUC), the volume of distribution central and peripheral (Vc & Vd), half-life (t½), therapeutic metabolism, clearance (CI), intercompartmental clearance (Q), therapeutic excretion, among others, and/or a combination thereof. For example, the information may be collected from the therapeutic's Clinical Pharmacology Review from the Office of Clinical Pharmacology (OCP) reviews of New Molecular Entity (NME) New Drug Applications (NDAs) and Original Biologics License Applications (BLAs), from the FDA.

As used herein, “cell or biologic parameters” can include, for example, cell type and subtype via expression of phenotypic markers, number of cells, viability, number of biomolecules expressed (e.g., cytokine or other receptors or chimeric antigen receptors (CARs) per cell), metabolic signature, or avidity and affinity of a biologic for a receptor, e.g., antibody.

In some embodiments, the system 110 may determine (i) one or more effective treatment therapies and/or (ii) an optimal treatment therapy based on the one or more determined effective treatment therapies. A treatment therapy may be considered an “effective” treatment therapy for a patient if the metrics for the therapy meet certain “effective” criteria. For example, the criteria may be designated or settable (variable) and refer to attributes such as: (i) the number of leukemic cells to achieve morphologic complete remission below a threshold (5%) in the BM or other more sensitive measure determination for MRD, (ii) the number of normal cells above a threshold related to hematological toxicity levels so as to reduce risk of BM failure and ensure recovery, (iii) the absolute number of cells in peripheral blood above a threshold so as to avoid cytopenias, and/or (iv) goals of care for the patient (such as disease curative intent, disease control, days of expected inpatient hospital stay for therapy and recovery, intent to maximize the outpatient quality of life during disease control, cost, among others, or any combination thereof). Each of these set criteria can relate to a goal of therapy for a patient.

In some embodiments, a treatment therapy may be considered “optimal” if the corresponding treatment information and metrics (i) are determined “most effective” by a treatment optimization module 128 and/or (ii) are optimized constraints to “optimal” criteria by the treatment optimization module 128. By way of another example, the system may optimize the “most effective” treatment by constraining the corresponding treatment/treatment combination, doses, and schedule to (i) a maximum cumulative dosage per day for each therapeutic and/or maximum number of administrations (e.g., infusion times) during treatment and (ii) minimum absolute number of neutrophils during treatment

### Example Pharmacokinetic/Pharmacodynamic Simulation Module

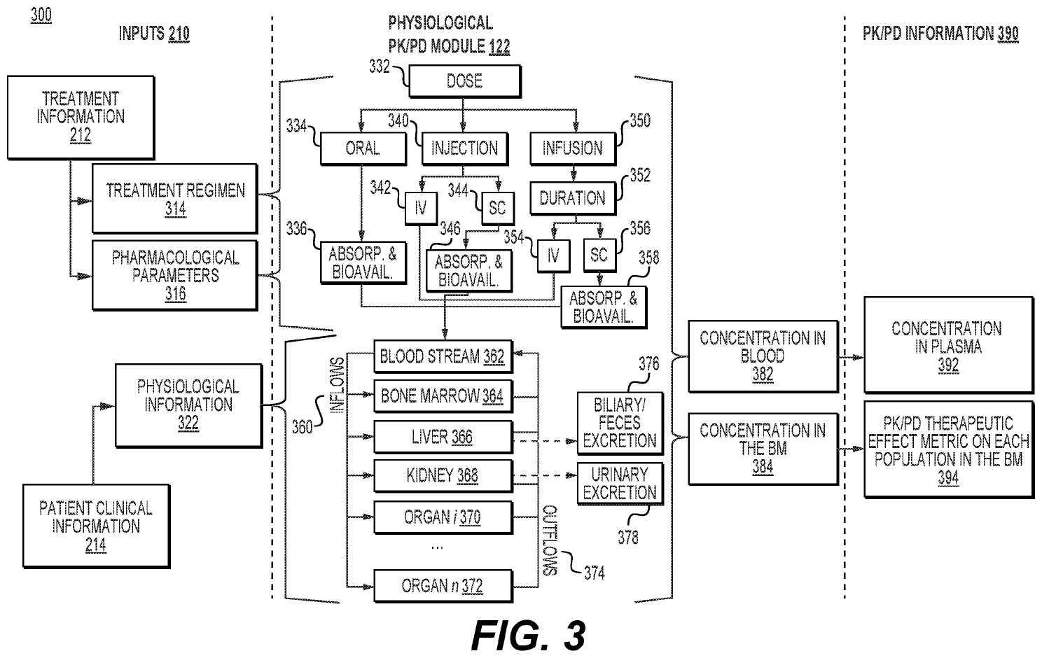

In some embodiments, the system 110 may include a pharmacokinetic/pharmacodynamic (PK/PD) module 122 configured to determine PK/PD therapeutic effect metric using the patient and treatment information for each therapy (e.g., mechanism of action) and regimen (e.g., treatment schedule). FIG. 3 shows an example implemnetatoin of the PK/PD module 122 according to embodiments.

Referring still to FIG. 1, the therapeutic effect metric may be employed in the stimulation and/or optimization to generate treatment metric(s). By way of example, the PK/PD therapeutic effect metric may include, or correspond to, the percentage of cells affected by therapeutics' action. In some examples, the PK/PD therapeutic effect metric may be determined, in the simulations, based on the PK module output, the concentration of the drug in the target organ, the pharmacodynamic parameters (such as the concentration of the drug to reach maximum effect), and the concentration of the drug at which the effect is 50% of the maximum. In some embodiments, the PK/PD module 122 may correspond to or incorporate features of the PK/PD module as discussed in, for example, Pefani E, Panoskaltsis N, Mantalaris A, Georgiadis MC, Pistikopoulos EN, Design of optimal patient-specific chemotherapy protocols for the treatment of acute myeloid leukemia (AML), Computers & Chemical Engineering, 2013, 57: 187-195, and Pefani E, Panoskaltsis N, Mantalaris A, Georgiadis MC, Pistikopoulos EN. Chemotherapy drug scheduling for the induction treatment of patients with acute myeloid leukemia. IEEE Trans Biomed Eng. 2014 July; 61 (7): 2049-56, which are each incorporated by reference in its entirety.

Referring back to FIG. 1, In some embodiments, the PK/PD module 122 may retrieve pre-stored PK/PD information (shown and referred to herein as “PK/PD Parameters” 142) to use in the PK/PD simulation to determine the metric, among other parameters described herein. The pre-stored PK/PD information may include a stored set of PK parameters, pharmacokinetic (PK) equations defining relationships among absorption, distribution, metabolism, and elimination of therapeutics, and pharmacodynamic (PD) equations defining a drug effect.

In some embodiments, for each treatment therapy provided to the PK/PD simulation, the PK/PD module 122 may determine a set of the metrics using the set of equations, patient information, and the corresponding treatment regimen information (e.g., standard clinical treatment and schedule data (e.g., drug, dose, dose duration, interval between doses, and the interval between successive treatment cycles and/or total treatment duration)).

In some embodiments, the PK/PD module 122 may operate on the PK/PD parameters 142 for each treatment therapy, for example, that is approved by the Food Drug administration (FDA) and/or is known (e.g., clinical trials). For example, the PK/PD parameters 142 may include or correspond to the equations and the treatment information for a corresponding treatment therapy. In the example, for a particular treatment therapy, the PK/PD module 122 may use the stored parameters 142 and the received patient information (e.g., proposed or current treatment plan) to determine the patient-specific metrics for that treatment therapy. In some embodiments, the PK/PD module 122 may determine the metrics using the stored equations and the parameters inputted by the clinician (e.g., a new novel treatment, one to be used in a clinical trial intended for the planning of the trial, etc.).

### Example Cell Population Simulation Engine

In some embodiments, the system 110 may include a population simulation engine 124 configured to use (i) modules 125 and 126, (ii) patient information (iii) treatment information to simulate a patient's disease state and/or response to a treatment therapy to quantitatively predict the patient's disease state and/or response to a treatment therapy. The PK/PD module 122 can be disabled in the simulation and its outputs omitted, for example, if the simulation is performed to quantitatively predict the patient's disease state without therapeutic intervention or when a different treatment therapy (e.g., a cell therapy) not having PK/PD is employed (see, e.g., FIG. 1B).

In some embodiments, the population simulation engine 124 may be configured to use stored modules 125 and 126 (i) based on patient information to simulate and quantify a prediction of disease state and/or (ii) based on patient information and treatment information to simulate and quantify a prediction of disease state and response to one or more treatment therapies. In some embodiments, the modules can account for recruitment from previous differentiation compartments, cell cycle stages, quiescence, and natural apoptosis to maintain homeostatic levels, death due to drug effects, and recruitment to the following differentiation compartment.

In some embodiments, for example, for leukemia, the population simulation engine 124 may include (i) abnormal (also referred to as sub-clonal) population modules 125 configured to simulate dynamic proliferation of each sub-clonal population and (ii) normal population modules 126 configured to simulate the dynamic hematopoietic process of differentiation (e.g., from stem cells to mature RBC, WBC, and L, in the BM and peripheral blood to death) in the target organ (e.g., BM), with or without treatment using the model according to the stored simulation Population Balance Module (PBM) parameters (e.g., average cell cycle times, cycling threshold, homeostatic absolute number of cells, etc.). In some embodiments, the population simulation engine 124 can also use the PK/PD parameters 142 to quantify a response to a therapeutic treatment. For example, the therapeutic's mechanism of action can determine which cell type will be affected by the therapeutic and in which cell cycle stage the treatment will act.

In some embodiments, the abnormal population modules 125 can include modules that simulate each sub-clone proliferation and death due to therapeutic effects according to their cell cycle time and sensitivity to the therapeutics (e.g., based on the aberrations and cell type), respectively. For example, aberrations may include but are not limited to gene point mutations or deletions, chromosomal structural re-arrangements (e.g., deletion, duplication, inversion, and/or translocation), epigenetic modifications, surface receptor expression/sensitivity, and cell action, among others or a combination thereof.

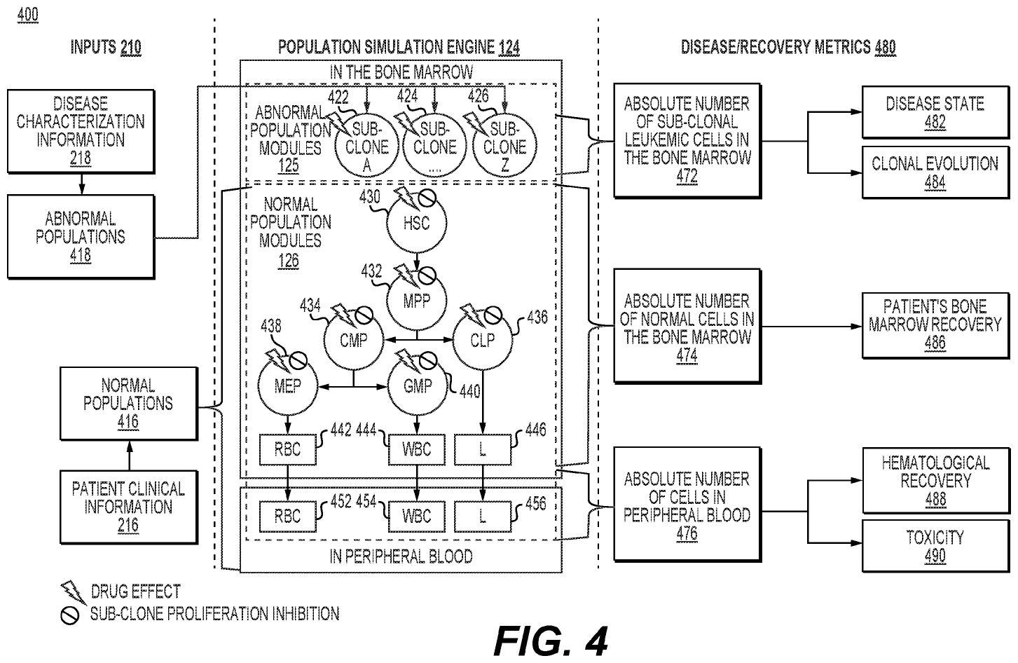

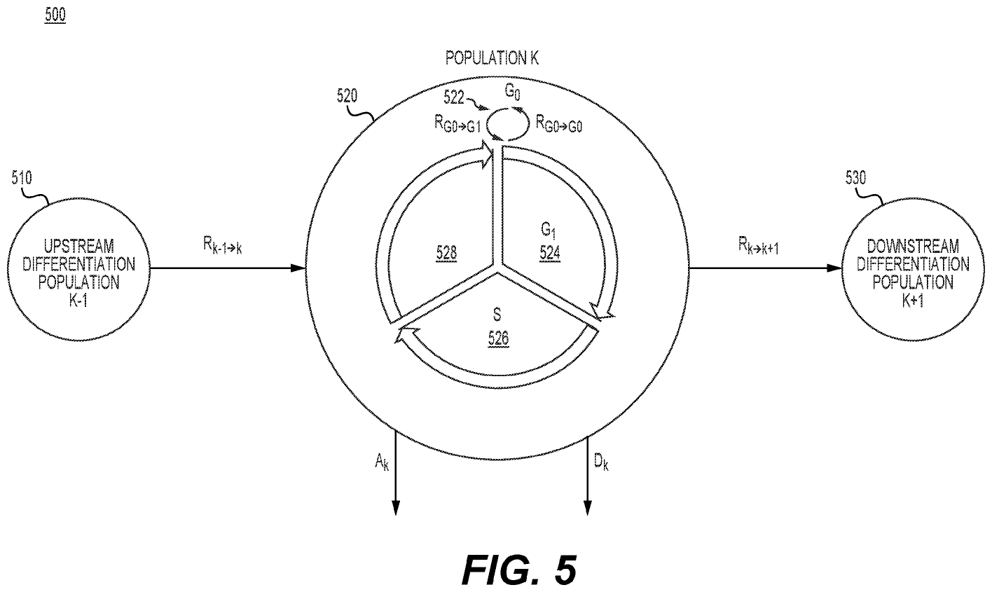

For example, the normal population model modules 126 can include modules for each cell compartment (e.g., cell type) according to a hierarchical hematopoietic differentiation tree model (e.g., as shown in FIG. 4), modules for each of a plurality of stages of the cell cycle phases for each compartment (e.g., as shown in FIG. 5), among others, or a combination thereof. The cell cycling times, recruitment rates, and apoptosis can be variable for each cell type compartment.

In some embodiments, using the patient information, the population simulation engine 124 can determine the number of normal and abnormal cells in each population compartment/module 125, 126 at specific time points during the treatment period following the dynamic hematopoietic process of differentiation from stem cells to mature RBC, WBC, and L, in the BM and peripheral blood, and also following the dynamic proliferation of each sub-clonal population in the target organ. In some embodiments, the population simulation engine 124 can incorporate the PK/PD treatment effect metric (e.g., percentage of cells affected in each sensitive population compartment) according to the corresponding treatment information (e.g., treatment schedule and therapeutic-specific mechanism of action) to simulate treatment. The population simulation engine 124 can determine which cell cycle stages and/or cell types of the normal and abnormal models can be affected by the treatment based on the therapeutic-specific mechanism of action.

In some embodiments, the population simulation engine 124 can determine one or more metrics, such as the absolute number of long-term hematopoietic stem cells (LT-HSC), short-term hematopoietic stem cells (ST-HSC), multipotent progenitors (MPP), common myeloid progenitors (CMP), common lymphoid progenitors (CLP), megakaryocyte-erythroid progenitors (MEP) and granulocyte-monocyte progenitors (GMP), and/or sub-clonal populations depending on patient tumor profile, and mature cells in all lineages in the BM and PB, and sensitive and resistant abnormal cells. In some embodiments, from these metrics, the engine 124 may determine additional metrics, such as disease burden or percentage of abnormal cells in the target organ (e.g., disease metric(s), which define the overall treatment outcome (e.g., disease metric can include CR (complete remission), PR (partial remission), RD (resistant disease, and/or Relapse-disease metric), percentage of each sub-clone in different time points during treatment, which define the sub-clonal evolution (e.g., disease metric(s)) and the absolute number of neutrophils in peripheral blood that determines if the patient achieved complete hematological remission (e.g., recovery metrics).

In some embodiments, the population simulation engine 124 may quantify disease state and/or patient response to a treatment by determining one or more disease metrics and/or recovery metrics using the model. For example, one or more disease and/or recovery metrics may include but is not limited to therapeutic concentration in blood, bone marrow recovery (e.g., normal stem cells, progenitors, precursors, and/or the absolute number of mature hematopoietic and immune cells), disease state (e.g., leukemic sub-clones absolute number and/or percentage), hematological recovery (e.g., the absolute number of mature hematopoietic cells, immune cells, and/or hemoglobin in the blood stream), among others, or a combination thereof. For example, the population simulation engine 124 can determine the number of residual viable normal and abnormal cells in each population compartment after treatment, predicting the dynamic multisystemic response to treatment, including: sub-clonal evolution, disease state, hematopoietic and immune response, and recovery in the BM and PB (also referred to as “hematological recovery”).

The system 110 can determine the level of cell death in each normal and abnormal population compartment during therapy and hematological recovery after each treatment cycle (e.g., defined by the schedule of administrations) in a dynamic manner (e.g., normal and abnormal absolute cell numbers in the BM and peripheral blood). In some embodiments, the engine 124 can use that information to determine disease and/or recovery metrics that relate to patient hematological, leukemia and immune response to treatment, patient recovery, disease state, clonal evolution, MRD, iRAEs, overall outcome hematological recovery, among others, or a combination thereof.

In some embodiments, the system 110 may include an optimization module 128 configured to: 1) select one or more effective treatment therapies based on effective criteria; and/or 2) optimize one or more effective treatment therapies by identifying a treatment therapy that constrains the maximum cumulative dose per day based on normal clinical practice and FDA guidance, to reduce treatment toxicity while improving outcomes. In some embodiments, the optimization module 128 may be configured to: 1) discard any additional and/or alternative treatment therapy that is contraindicated for certain comorbidities; 2) determine out of the remaining therapies the engine 124 to generate simulations of cell cycles for the patient to determine one or more metrics; and 3) using the metrics from the generated simulations, select the most effective treatment (lowest percentage of abnormal cells, fastest recovery of the absolute number of normal cells (shortest period of time to reach that normal range) and highest absolute number of neutrophils during treatment). In some embodiments, the optimization module 128 may also be configured to optimize the “most effective” treatment combination, doses, and schedule to constrain the maximum cumulative dosage per day (or overall) for each therapeutic, maximum/minimum infusion times, and minimum absolute number of neutrophils during treatment, to maximize the number of normal cells while minimizing the number of abnormal cells in the BM, to ensure treatment effectiveness and patient recovery.

In some embodiments, the system 110 may include a metric reporting module 130 configured to generate a report based on the one or more patient, disease, treatment, and/or recovery metrics for each treatment therapy, including the optimal treatment therapy. The report and the corresponding treatment information can be stored in the database 146, along with the associated patient and treatment information, provided on a display (e.g., user computing system 160), among others, or a combination thereof. For example, the reports may include quantitative visualizations of the metrics determined by the population simulation engine 124 for each treatment therapy simulated (including no treatment therapy). For example, the visualizations may include daily disease burden (e.g., percentage of leukemic cells in the bone marrow), number of normal cells (patient recovery), and abnormal sub-clonal cells (disease status) in the bone marrow and peripheral blood. WBC, RBC, and lymphocytes in peripheral blood, hemoglobin concentration, and/or Absolute Count of Neutrophils (ACN). For example, these visualizations can show hematological recovery and treatment toxicity to a specific treatment, which can be used by a clinician for evaluation.

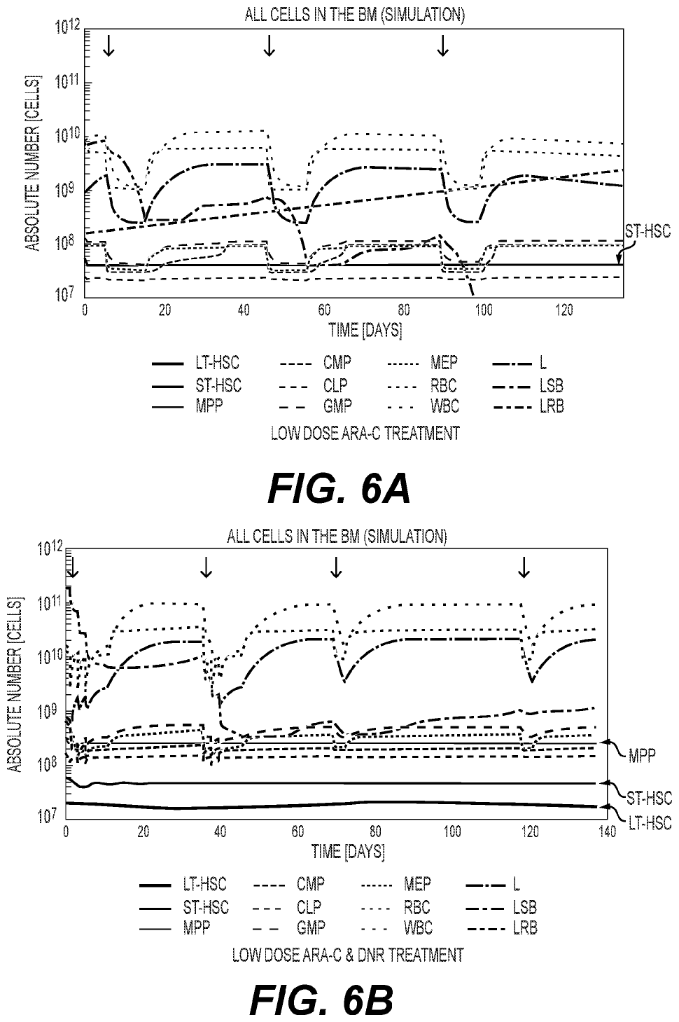

For example, FIGS. 6A-14 show examples of quantitative visualizations that may be generated using the methods and systems according to embodiments. This way, a clinician can analyze the pros and cons of each treatment with respect to a patient's expressed treatment goals, outcome, recovery, and cost.

In some embodiments, the system 110 may include an assessment module 132 configured to evaluate the patient-specific model-based simulations based on the patient's (actual) response to the administration of the treatment therapy at one or more time points during the treatment period. For example, the module 132 may compare the metrics determined from the simulations to the metrics collected by clinical assessments performed on the patient after administration of the corresponding treatment therapy to determine whether the patient's response corresponds to the simulations. If the assessment module 132 determines the patient response is not within the thresholds of the simulation metrics, the assessment module 132 may determine patient-specific parameters (e.g., cell cycle time and therapeutic sensitivity for sub-clonal populations, and/or neutrophils recovery rate) that can be inputted into the population stimulation engine 124 to update the model and run additional simulations to determine metrics and any changes to the administered treatment therapy.

Although the systems/devices of the environment (e.g., 100A) are shown as being directly connected, the system 110 may be indirectly connected to one or more of the other systems/devices of the environment 100A. In some embodiments, the system 110 may be only directly connected to one or more of the other systems/devices of the environment 100A.

It is also to be understood that the environment 100A may omit any of the devices illustrated and/or may include additional systems and/or devices not shown. It is also to be understood that more than one device and/or system may be part of the environment 100A although one of each device and/or system is illustrated in the environment 100A. It is further to be understood that each of the plurality of devices and/or systems may be different or may be the same. For example, one or more of the devices may be hosted at any of the other devices.

### Example System #2

FIG. 1B depicts another example of a system environment 100 (shown as 100B) for generating patient-specific simulations using the mathematic, mechanistic, deterministic and modular model and related information (e.g., disease and patient with or without treatment information) in a target organ (i) to predict a disease state (e.g., without treatment information) by determining one or metrics, and/or (ii) to predict a patient response to a current or simulated cell therapy. The simulation may be used to determine a treatment therapy, therapy adjustments, or based on the metrics according to some embodiments.

In the example shown in FIG. 1B, the population simulation engine 124 is configured to receive cell therapy parameters 123 to perform the mechanistic simulations to predict the patient's personalized disease state and/or response to cell therapy treatment. The simulation may be used to determine an optimal treatment according to embodiments or to adjust the therapy. Examples of cell therapy parameters may include but is not limited to the number of provided types of T cells, NK cells, monocytes/macrophages, different types/subtypes of T cells and NK cells or other cell types, or engineered cell therapies such as CART or CAR-NK cells, etc. that are, e.g., provided for to the treatment of patients with stem cell transplants and prediction of recovery kinetics, stem cell mobilization protocols and other relevant treatment protocols for cell therapy and graft vs. host disease.

In the example shown in FIG. 1B, the system 110 may include simulation modules configured to determine an optimal immune cell number to effect immune control of the tumor.

### Example Method of Operation

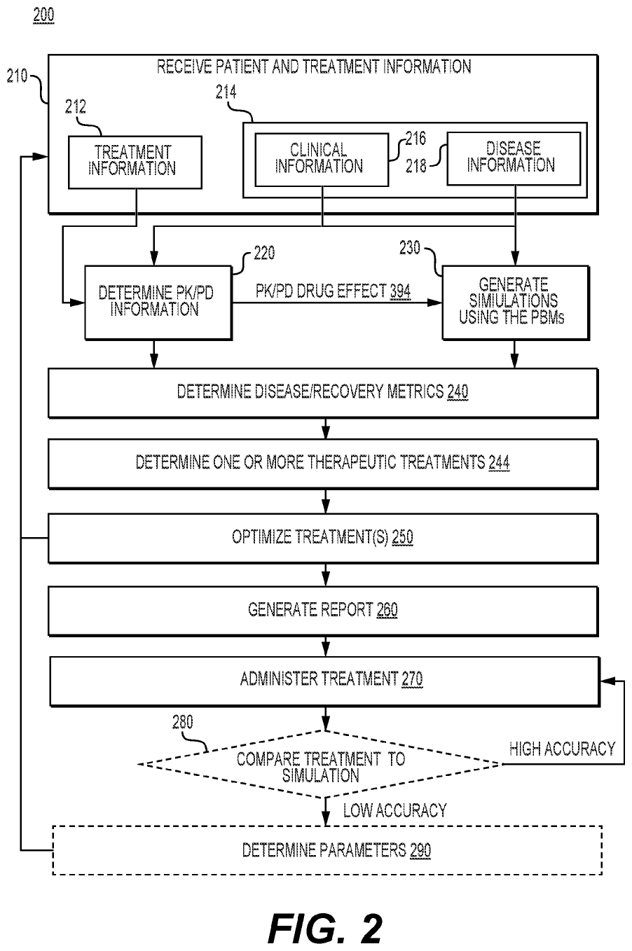

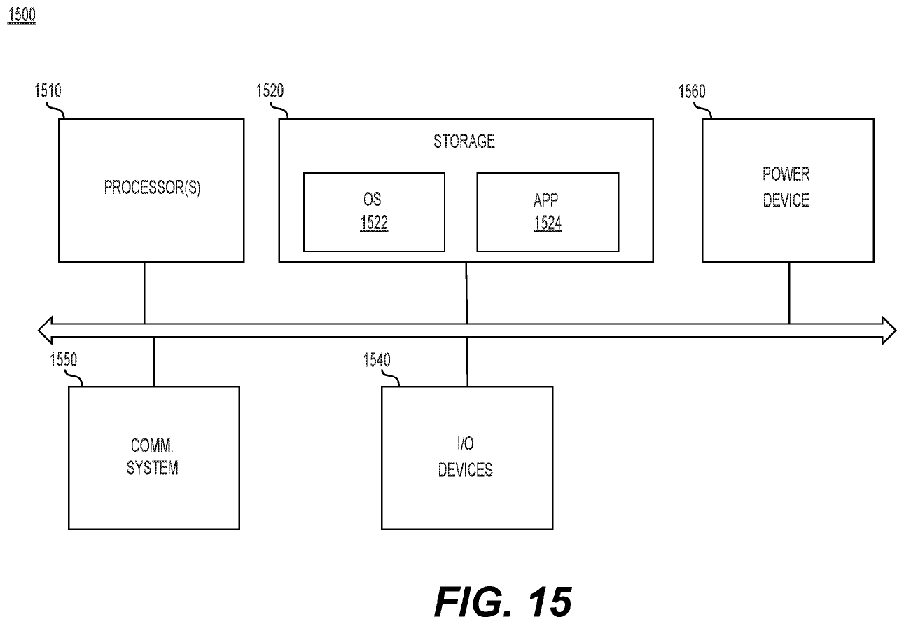

FIG. 2 shows a flow chart 200 illustrating examples of a method of determining an optimal treatment therapy according to certain embodiments. Operations described in flow chart 200 may be performed by a computing system, such as the system 110 described above with respect to FIG. 1A or a computing system described below with respect to FIG. 15. Although the flow chart 200 may describe the operations as a sequential process, in various embodiments, some of the operations may be performed in parallel or concurrently. In addition, the order of the operations may be rearranged. An operation may have additional steps not shown in the figure. In some embodiments, some operations may be optional. Embodiments of the method may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium.

Operations in the flow chart 200 may begin at block 210, the system 110 may receive patient and treatment information, in some embodiments. For example, the system 110 may receive treatment information 212 (e.g., therapeutic dose, administration route, and method, and/or schedule), corresponding to that one or more treatment therapies, for example, via the system 160, by the clinician inputting the treatment regimen parameters for a corresponding treatment therapy, among others or a combination thereof; retrieve the treatment information 212 from a stored database, healthcare information systems, etc.; among others. The PK/PD parameters associated with the treatment may be provided by the clinician and/or retrieved from the PK/PD parameters database 142. In some embodiments, operations may also be initiated when the treatment therapy is optimized at block 250 or when the parameter(s) are updated at block 290 based on monitoring at block 280, according to embodiments.

In some embodiments, the system 110 may receive patient information 214 (for an individual patient), such as clinical information 216, and the disease characterization information 218, for example, via the healthcare information system 170.

In some embodiments, the system 110, at block 220, may determine PK/PD information for each treatment therapy. For example, FIG. 3 shows a flow chart 300 illustrating an example of a method to determine PK/PD information (i.e., outputs 390) for each treatment therapy (identified in step 210) by the PK/PD module 122 to be used by the population simulation engine 124 according to certain embodiments. In some embodiments, the PK/PD information (i.e., outputs 390) may be generated using the PK/PD parameters (142) for each treatment therapy and clinical information 216 for the patient. For example, using the method shown in FIG. 3, the PK/PD information (e.g., outputs 390) can be determined for each treatment therapy to be evaluated. In some embodiments, the treatment information 212, such as the therapeutic pharmacological information 316 and treatment regimen 314 parameters, and the patient clinical information 216, such as the patient's physiological parameters 322, may be inputted (210) by the clinician and/or retrieved by the system 110 from a stored database, healthcare information systems, etc.

In some embodiments, the treatment regimen parameters 314 may include dose 332, administration method (e.g., oral 334, bolus injections 340, and/or infusion 350), administration length (e.g., for infusion, duration 352), administration route (e.g., intravenous (IV) 342, subcutaneous (SC) 344 and/or oral (not shown)), among others, or any combination thereof. Therapeutics' pharmacological parameters 316 may include but are not limited to absorption and/bioavailability 336, 346, 358, maximum plasma concentration (Cmax) (not shown), time until Cmax is reached (Tmax) (not shown), area under the concentration-time curve (AUC) (not shown), the volume of distribution central and peripheral (Vc & Vd), half-life (t½), therapeutic metabolism, clearance (Cl) and intercompartmental clearance (Q).

In some embodiments, using the inputs 210, the PK/PD module 122 may first determine the inflow 360 from the dose 332 and duration of the administration (e.g., bolus or infusion 350) information 352 and the therapeutic's absorption and bioavailability 358. The inflow 360 may first be distributed to the blood stream 362. After, the therapeutic in the blood stream 362 may be distributed into different organs (e.g., bone marrow (BM) 364, liver 366, kidney 368, other organs i-n (370-372), etc.) to determine outflow 374, for example, where it is metabolized and excreted in the biliary tract, feces excretion (e.g., biliary/feces extraction 376) and/or urine (e.g., urinary excretion 378). The remaining of the therapeutic(s) will eventually reach the target organ, such as the BM 364, where the therapeutic(s) is going to affect the normal and abnormal cells based on pharmacokinetic parameters.

The PK/PD module 122 can use the therapeutic dose 332 and the administration method and route (e.g., oral 334, injection 340, and/or infusion 350) coupled with the therapeutic absorption and bioavailability (336, 346, and/or 358) to determine the concentration of the therapeutic in the blood stream 382. Then, the PK/PD module 122 can use the patient physiological information 322 to determine the organ volume and blood volume in each organ, and the therapeutic-specific pharmacological information 316 to determine the therapeutic's metabolism, clearance in each organ (BM 364, liver 366, kidney 368, lungs, guts, muscle, heart, (e.g., 370-372), etc.) and the residual therapeutic concentration in the blood stream 382 after entering these organs. The organ volume and blood volume, and the residual therapeutic concentration in the blood stream 382 can be used to determine the concentration of the therapeutic in the target organ (BM) 384. The PK/PD module 122 can determine the “PK/PD Therapeutic Effect” 394 from the therapeutic concentration 382 using PD equations and pharmacological parameters 142, such as effective concentration (Emax) and half maximal effective concentration (E50), which determine the percentage of cells affected by the therapeutic in a sensitive population compartment (target population for targeted therapeutics). The population compartment sensitivity can be based on the pharmacological therapeutic-specific mechanism of action (targeted cell cycle phase or population compartment expressing a certain biomarker). The PK/PD module 122 may calculate therapeutic concentration in the peripheral blood 382 (inflows 360). Using the therapeutic concentration in the peripheral blood and the patient information (e.g., the fraction of plasma), the therapeutic concentration in plasma 392 may be determined. For example, the physiological parameters 142 may be used to calculate organ size and therapeutic metabolism, and the pharmacological parameters 316 may be used to calculate therapeutic metabolism, urinary excretion 378, and biliary/feces excretion 376 to determine the therapeutic concentration in each organ (outflows) 374, in particular, in BM (384).

In some embodiments, the system 110, at block 230, may generate simulations of each normal compartment and abnormal sub-clonal populations using the modules 125 and 126 with each treatment therapy (e.g., corresponding PK/PD Therapeutic Effect 394 determined in step 220) and/or without treatment (steps 212 and 220 can be omitted). FIG. 4 shows a flow chart 400 illustrating examples of a method to generate the simulations of abnormal and normal cells using the abnormal and normal modules 125, 126 for each treatment therapy by the engine 124 according to certain embodiments. In this example, the treatment therapy is for treating leukemia, and the target organ is BM.

For example, as shown in FIG. 4, the number of normal and abnormal cells in each compartment of the corresponding normal populations 416 and abnormal populations 418, for example, determined from the BM aspirate and biopsy assessment included in the patient clinical information 216 and disease characterization information 218 (received in step 210), may be inputted as initial conditions.

In some embodiments, the number of cells in the target organ (e.g., BM) can be early immature (stem and progenitor) cells to more mature cells of the blood lineages found in the aspirate/biopsy sample. For example, the normal populations 416 in the BM can be processed as interconnected population compartments based on the human hematopoietic differentiation tree model starting with hematopoietic stem cells (HSC) 430, followed by multipotent progenitors (MPP) 432, common myeloid progenitors (CMP) 434, common lymphoid progenitors (CLP) 436, megakaryocyte-erythroid progenitors (MEP) 438, and granulocyte-monocyte progenitors (GMP) 440, and mature red blood cells (RBC) 442, white blood cells (WBC) 444, and lymphocytes (L) 446. In this example, each cell type, HSC 430, MPP 432, CMP 434, CLP 436, MEP 438, GMP 440, RBC 442, WBC 444, and L 446 can represent a population compartment.

In some embodiments, the mature populations in peripheral blood can be RBC 452, WBC 454, and lymphocytes (L) 456. In this example, the populations can be cycling (circles) (e.g., sub-clone(s) 422, 424, and 426, HSC 430, MPP 432, CMP 434, CLP 436, MEP 438, or GMP 440) or non-cycling (rectangles) (e.g., RBC 442, WBC 444, or L 446). If the population is cycling, the compartment may be represented by FIG. 5, to simulate quiescent cells (dormant cells waiting to enter the cell cycle), cells progressing in stages of specific cell phases and between cell cycle phases, entering the compartment from previous compartments (differentiation), differentiating into downstream differentiation stages (leaving the compartment) and dying due to apoptosis or drug effects (leaving the compartment).

For example, the engine 124 can process the normal populations using the module(s) 126 according to the human hematopoietic differentiation tree model. As shown in FIG. 4, according to the tree model, the engine 124 can process the cells using the modules 126 so that the normal cells differentiate into lineage-restricted progenitors that undergo extensive proliferation and differentiation to produce terminally differentiated and functional hematopoietic cells. For example, in the tree model as shown in FIG. 4, downstream to the multi-potent compartment can be two oligo-potent compartments, CMP 434 and CLP 436; CMP 434 can give rise to GMP 440 and MEP 438; and CLPs 436 would differentiate into Pro-T, Pro-B and Pro-NK cells that will produce mature T cells, B cells and natural killer cells (L compartment 446). GMPs 440 and MEPs 438 can differentiate into mature granulocytes (basophils, eosinophils, and neutrophils), macrophages and dendritic cells (WBC compartment 444), and megakaryocytes and erythrocytes (RBC compartment 442), respectively. Each cell type represents a “differentiation or cell-type compartment.” By way of another example, hemoglobin concentration in PB can be determined based on the personalized Mean Corpuscular Hemoglobin Concentration (MCHC), which is included in the patient clinical information and represents the average concentration of hemoglobin per RBC 442.

In some embodiments, using the modules 125 and 126 and the PK/PD Therapeutic Effect 394, the engine 124 can determine the number of the abnormal and normal cells to be affected by therapeutic agents (lightening symbol in FIG. 4) and simulate the growth of normal cells to be inhibited by leukemic populations with the same immunophenotyping profile that defines the cell type (0 in FIG. 4). The engine 124 can use the number of abnormal and normal cells to determine the absolute number of sub-clonal leukemic cells in the BM 472, the absolute number of normal cells in the BM 474, and the absolute number of cells in PB 476.

In some embodiments, the engine 124 may have a different stored homeostatic level for each cell type compartment of normal cells as stored parameters 144. By way of example, each compartment in the BM may have the following homeostatic level relationship: 0.01% LT-HSC, ST-HSC 0.03%, MPP 0.17%, CMP 0.42%, CLP 0.08%, GMP 0.38%, MEP 0.35%, WBC 59.4%, RBC 25% and L 13.1%.

In some embodiments, the engine 124 may use different cell cycling times, recruitment rates, and apoptosis rates in the modules 126 based on the stored homeostatic level for the compartment corresponding to that module. By way of example, when the engine 124 determines that the number of cells in the differentiation compartment is lower than its corresponding stored homeostatic level, the engine 124 can cause recruitment of quiescent cells to cycle, reduce cycling time to increase proliferation and increase recruitment from the up-stream compartment. For example, for the cycling time, the cell cycle time can be reduced, based on the number of cells in the compartment, to increase proliferation in the compartment and produce more cells. The recruitment rates from previous compartments and quiescence can be increased exponentially by a stored factor to induce differentiation into the unbalanced compartment.

By way of another example, when the engine 124 determines that the number of cells in the differentiation compartment is higher than its homeostatic level, the engine 124 can cause the cells in the compartment to re-establish their quiescence level, cycling times, and recruitment rates from up-stream compartments, and increase apoptosis. For example, the cell cycle times and the recruitment rates can be set to their corresponding stored average values for that compartment/stage/module 126. The apoptosis rate may also be increased by a stored factor corresponding to that compartment/module.

For example, for leukemia, the abnormal population compartments 422, 424, 426, such as the number of cells 418 in each sub-clonal population and the number of sub-clonal populations in the bulk tumor (e.g., sub-clone A 422, sub-clone B, sub-clone C, sub-clone . . . 424, sub-clone Z 426), may be provided by the disease characterization information 218. The grouping of the abnormal populations 418 into respective compartments may be based on the cell type and concurrent aberrations they carry, which are determined by tumor biopsy analysis, for example, using a tumor biopsy analysis system, such as Next Generation Sequencing (NGS), whole genome sequencing (WGS), whole exome sequencing (WES), single-cell omics or multi-omics or similar genomics platform), among others, or a combination thereof. For example, the results of these analyses may define the number of cells in each sub-clonal population and the number of sub-clonal populations in the bulk tumor (e.g., sub-clone A 422, sub-clone B, sub-clone C, sub-clone . . . 424, sub-clone Z 426 in which Z can be any number).

FIG. 5 shows an illustration of the cell cycle phases through which the cells in each compartment of the cell type (e.g., 430, 432, 434, 436, 438, 440, 442, 444, 446) shown in FIG. 4 can proliferate according to the simulation generated by the engine 124 using the modules 125 and/or 126. In FIG. 5, each cell-cycle phase (e.g., G₁ 524, S 526, G₂/M 528) can be implemented to track cells in the various cell cycles in bins. The engine 124 can generate a simulation, e.g., in the respective modules 125 and/or 126, that models and represents the cycling in the corresponding cell cycle phase compartment for normal and/or abnormal cell types via their corresponding data object.

In each cell cycle phase simulation, e.g., shown in the example of FIG. 5, the proliferating normal cell type under analysis (e.g., “HSC” 430, “MPP” 432, “CMP” 434, “CLP” 436, “MEP” 438, “GMP” 440, “RBC” 442, “WBC” 444, “L” 446) can be defined and implemented with a population compartment that is populated by the respective bins corresponding to the cell cycle phases. In this example, the sub-clonal populations (422, 424, 426) can be considered independent “K” populations without differentiation (no K−1 or K+1 compartments 510, 530) and can be implemented with separate compartments. In this example shown in FIG. 5, the gap or Growth “1” Phase (G₁) 524 can be considered the first of four phases of the cell cycle that takes place in eukaryotic cell division in which the cell grows physically larger, copies organelles, and makes the molecular building blocks it will need in later phases. The synthesis Phase(S) 526 can be considered the second of the four phases in which the cells synthesize a complete copy of the DNA in its nucleus, the cells duplicate a microtubule-organizing structure called the centrosome, and the centrosomes help separate DNA during the mitotic phase (M) 528. The Second Gap or Growth 2 Phase (G₂) 528 can be considered the third of the four phases in which the cells make proteins and organelles and begin to reorganize their content in preparation for the next phase, mitosis. The Mitosis phase (M), also shown in 528, can be considered the fourth of the four phases in which the cells divide their copied DNA and cytoplasm to make two new daughter cells.

In some embodiments, the engine 124 can cause each normal population compartment “K” 520, for example, in the BM and PB, to be maintained so that it equals the stored human physiological homeostatic cell type percentage for that compartment (e.g., for 430, 432, 434, 436, 438, 440). The parameter can be established based on the average percentage of each cell type in the BM and PB for humans. For example, if the number of cells in the population compartment K 520 is low, the engine 124 can recruit cells from the previous compartment (K−1) 510 at rate R_{k-1→k}, or from the quiescence cell cycle state (G₀) 522 into the cell cycle (G₁ 524, S 526, and G₂/M 528) to produce two daughter cells at rate R_{G0→G1}, to increase their cell number in the compartment. By way of another example, if the number of cells is higher than the homeostatic level, the engine 124 can determine that the cells will die due to apoptosis at the rate Aₖ or enter G₀ 522 at the rate, R_{G1→G0}, to stay dormant. Also, the engine 124 can cause continuous differentiation of cells in the population compartment “K” 520 into downstream populations (K+1) 530 at rate R_{k→k-1} to produce functional cells in the bloodstream, which eventually die according to the documented mature cells lifespan. Finally, the engine 124 can determine that each population dies due to “PK/PD Therapeutic Effect” 394 at rate Dₖ depending on the therapeutic mechanism of action, concentration in the target organ, and/or sensitivity of the target cell population.

In some embodiments, the number of stages of the cell cycle can be fixed for each phase. In some embodiments the cell cycle transitions within and between the stages and/or phases of the cell cycle, G₁ 524, S 526, and G₂/M 528, can depend on the concentration of cyclin E, DNA, and cyclin B, respectively.

For example, in use, within the Population K 520 simulation, the engine 124 can model the cells, e.g., in the respective compartments, to move into the next cell type (e.g., 430, 432, 432, 436, 438, 440, 442, 444, 446) based on their cell cycle state and progression in the individual cell cycle phase simulation. In the Population K 520 simulation, the engine 124 models cells as exiting the dormant or quiescent state (G₀) 522 and entering the Growth “1” Phase G₁ 524 in response to a determined increase in cyclin E concentration. The engine 124 can simulate the production of Cyclin E during the G₁ phase 524; the concentration of Cyclin E would eventually peak, and the simulation then transitions from the current cell-cycle phase to the S phase 526. The simulation evaluates the binding of the Cyclin E primarily to CDK-2, activating DNA duplication mechanisms. When the engine 124 determines from the simulation of DNA concentration that DNA synthesis simulation is considered complete, the simulation can direct the current cell phase to transition from the end of the S 526 phase to the G₂/M 528 phase. In the G₂/M 528 phase, the engine simulates the production of Cyclin B via a cyclin B concentration estimator module. Once the engine 124 determines that the cyclin B concentration reaches a concentration threshold, the simulation models the cyclin B to bind to CDK-1 resulting in the current cell cycle stage transitioning to the M 528 phase. The simulation can model cyclin E minima to represent the baseline expression at the beginning of the cell cycle phase, while the stored cyclin threshold values can be used to determine the average cyclin level at which the simulation determines that the cells would move to the next phase. The engine 124 can use a constant value for the cyclin E production rate in the G₁ 524 phase. The engine 124 can simulate the DNA production rate as a lineal function based on pre-defined normalized results. The engine can simulate a constant cyclin B production rate during and throughout G₂ 528 phase with a concentration plateau at transition. The engine 124 can determine a transition probability value for the likelihood of a cell at a particular stage in the cell cycle phase moving to the next phase. See, for example, Fuentes-Garí M, Misener R, Garcia-Munzer D, Velliou E, Georgiadis MC, Kostoglou M, Pistikopoulos EN, Panoskaltsis N, Mantalaris A. A mathematical model of subpopulation kinetics for the deconvolution of leukaemia heterogeneity. J R Soc Interface. 2015 Jul. 6; 12 (108): 20150276. doi: 10.1098/rsif.2015.0276. PMID: 26040591; PMCID: PMC4528591, and García Münzer DG, Kostoglou M, Georgiadis MC, Pistikopoulos EN, Mantalaris A (2015) Cyclin and DNA Distributed Cell Cycle Model for GS-NSO Cells. PLOS Comput Biol 11 (2): e1004062. https://doi.org/10.1371/journal.pcbi.1004062, which are each incorporated by reference in its entirety.

By way of example, the engine 124 may cause a number of cells per stage to move as follows. For G₀ 522 and the first G₁ phase 524, after mitosis 528, the engine 124 may cause two daughter cells to move from G₂/M 528 to the first stage in G₁ 524. In this phase, the engine 124 may simulate the recruitment of cells from and to G₀ 522 in response to demand-adapted and dynamic entry into and exit out of the cell cycle over time. The engine 124 may simulate the cells staying in the cycle, moving to the next stage, or moving to the next cell cycle phase, depending on their corresponding transition probability. Also, the engine 124 may simulate cells recruited from the upstream compartment moved into the first stage in G₁ 524. Cells in this stage can also be recruited to differentiate into downstream differentiation compartments. The engine 124 may determine the death of the cells in this stage via apoptosis (i) if the number of cells in the compartment exceeds the homeostatic level and (ii) due to the effect of therapeutics that affect DNA replication.

In this example, stages in G₁ 524 can include cells (i) coming from the previous stage, (ii) moving to the next stage or moving to S 526, (iii) coming from the upstream compartment, (iv) differentiating into downstream compartments, (iv) dying via apoptosis, and (v) dying due to the effect of therapeutics that affect DNA replication. In the first stage of the S 526, the engine 124 can cause (i) cells progressing from G₁ 524 to move into this stage 526 and (ii) cells progressing in the S 526 or cell cycle, dying due to apoptosis or therapeutics' effect move out of this stage.

In this example, for the first stage of the S 526, the engine 124 can cause (i) cells progressing from G₁ 524 to move into this stage and (ii) cells progressing to the S 526 or cells dying due to apoptosis or therapeutics' effect to move out of this stage. For the stages of the S 526, in the progression of S 526, the engine 124 can cause cells to (i) move from the previous stage, (ii) move to the next stage, (iii) die due to apoptosis, or (iv) die due to the effect of therapeutic drugs that affect DNA replication.

Also, in this example, for the first stage of the G₂/M 528, the engine 124 can cause (i) cells progressing from S 526 to move into the first stage of the G₂/M 528, (ii) cells progression in the G₂/M 526 move to the next stage, and (iii) cells progressing to G₁ 524, to produce two daughter cells that is then moved to G₁ 524 first stage. And, if the number of cells in the compartment is higher than the homeostatic level, the simulation can model the cells as dying due to apoptosis.

In some embodiments, the engine 124 can cause the mature cells, RBC 442, WBC 444 and L 446, to be derived from MEP 438, GMP 440, and CLPs 436 cycling progenitors, respectively, in the module 126, as shown in FIG. 4. The RBC 442, WBC 444, and L 446 in the BM can migrate to the PB (RBC 452, WBC 454, and L 456) depending on the recruitment of these cells from PB based on their homeostatic level, which corresponds to normal concentration in the PB for a human. For example, the WBC 452, e.g., neutrophils, dendritic cells, and macrophages, can be defined as differentiated cells in circulation in PB; these cells come from WBC 444 in the BM.

Like the normal cells, the engine 124 can also simulate the cycling of abnormal cells 418 through the phases and stages within the phases, as shown in FIG. 5 using the model 125. In some embodiments, the number and percentage of each sub-clonal population (e.g., 422, 424, 426) can be characterized by the concurrent mutational aberration that the sub-clone carries and specific cell-type biomarkers. For example, this information can be collected from BM or PM assessments performed at the facilities of the healthcare provider (e.g., using Next-Generation Sequencing and/or Whole Genome/Exon Sequencing, and/or single cell multi-omics' analysis, among others) and as expected for the standard of care in such patients. In this example, the engine 124 can use this information 218 to define the number of abnormal cells (e.g., the sum of leukemic sub-clones, such as sub-clone A 422, sub-clone . . . 424, and sub-clone Z 426) in the BM 472, as shown in FIG. 4. In the simulation, each of the sub-clonal populations (422, 424, and/or 426) can be considered to be independent abnormal population modules 125. For each sub-clonal population 422, 424, 426, the dynamic cell number can be calculated based on their cell type and aberrations. This information (e.g., cell type and aberrations) can be used by the engine 124 to define their average cell cycle time, activated metabolic pathways, and interaction with their microenvironment and/or niche (e.g., neighboring cells, cytokines, chemokines, soluble factors and/or structural components). The engine 124 can further determine the disease and/or recovery metrics 480 (e.g., disease state 482, proliferation rate, and/or clonal evolution 484) in response to the specific therapeutic, using the average cell cycle time, activated metabolic pathways, and interaction with their microenvironment or niche. For example, for leukemia and the BM as the target organ, the microenvironment may include but is not limited to neighboring cells, cytokines, chemokines, soluble factors, and/or structural components, which can be provided by a bone marrow assessment.

For example, the cell cycling time of each sub-clone can depend on the aberrations the sub-clonal population carries, mutations, or chromosomal structural re-arrangements (deletion, duplication, inversion, or translocation). The specific group of aberration(s) that define the sub-clones 422, 424, and/or 426 also define the cell cycling time and therapeutic-specific sensitivity of that sub-clone to targeted therapeutics. For example, the therapeutic-specific sensitivity to targeted therapies can depend on immunophenotype classification (expression of a targeted biomarker) or activity/kinetic classification. The sub-clones 422, 424, and/or 426 can be considered as a part of the cell number of the compartment that shares the same cell-type-specific biomarkers, thereby impacting proliferation, differentiation, and apoptosis of the normal cells in that compartment (for example, decreasing cell cycle time and recruitment from previous compartments and quiescence, and increasing apoptosis). This can allow the calculation of the detrimental effect on the production of mature and functional normal cells in the BM and PB. The modular incorporation of clones can allow the systems and methods of the disclosure to capture the initial intra-patient tumor heterogeneity and dynamically predict sub-clonal evolution, overall tumor burden, disease evolution, and patient outcomes.

In some embodiments, the engine 124 can cause each abnormal sub-clonal population 422, 424, and/or 426 to cycle through the simulation in the module 125 illustrated in FIG. 5, like the normal cells. In the simulation for abnormal sub-clonal populations (422, 424, 426), the engine causes the cells to only be recruited from the dormant or quiescent state (no recruitment from previous compartments) into the cell cycle. The engine 124 can also cause no differentiation. In some embodiments, the cell cycling time for each sub-clone can depend on the aberrations that the sub-clone carries. For example, the cell cycling time for each sub-clone (characterized by cell type and aberrations) can be stored in the parameters 144.

In some embodiments, the number of normal and abnormal cells used in the simulations by the engine 124 can depend on the concentration of the therapeutic in the BM 384 (determined by the PK/PD module 122), the effect 394 of the therapeutic on the normal and abnormal populations (determined by the PD equation), sensitivity to the specific therapeutic based on cell-type (normal cells) and cell-type plus aberrations (abnormal cells) (for targeted therapies, and the cell-type and cell cycle phase for chemotherapy.

After the engine 124 generates simulations using the modules 125 and 126 and the parameters (e.g., cell cycle time, cellular metabolism, interaction with their microenvironment, kinetics, therapeutic sensitivity, etc.) specific to the patient are performed for the treatment period/course, the population engine 124 can determine one or more disease and/or recovery metrics 480, such as the absolute number of cells in each sub-clonal population compartment 472 and an absolute number of cells in the normal population compartments (e.g., 430, 432, 434, 436, 438, 440, 442, 444, 446, 450, 452, and/or 454) in the BM 474 and PB 476, with or without the administration of therapy according to treatment information and/or PD therapeutic effect inputted by the clinician/user.

At block 240, the engine 124, can determine one or more disease and/or recovery metrics 480 for each treatment therapy simulated and/or one more disease and/or recovery metrics 480 without treatment therapy. The one or more metrics may include BM recovery 486 and/or PB recovery 488 (e.g., the time point when normal cells and hemoglobin are above a certain threshold), clonal evolution 484 (evolution of the sub-clonal population proportion within the tumor), disease state metric 482 (e.g., percentage of abnormal cells), treatment toxicity levels 490 (e.g., using therapeutic in plasma 392), among others, or a combination thereof.

In some embodiments, at block 244, the system 110 may determine a target treatment therapy of one or more therapeutics out of the simulated therapeutics based on the one or more metrics determined at block 240. By way of example, the target treatment therapy out of the therapies simulated may be determined by comparing the metrics for one or more therapeutics to treatment criteria. The treatment criteria may include, for example, measurement of disease state, MRD, clonal evolution, absolute number of neutrophils in PB, expected time in hospital, and expected other relevant toxicities (e.g., hemoglobin level). Optimal treatment may be determined by the greatest reduction of disease state and/or MRD, and the limitation of clonal evolution. In other examples, a clinician may select the target treatment therapy based on the metrics/simulations.

In some embodiments, at block 250, the system 110 may optionally optimize the target treatment and/or one or more treatment therapies analyzed at block 240 (e.g., treatment information 212), for example, selected by the clinician. For example, the treatment optimization module 128 may determine one or more effective therapies based on the effective criteria. In some embodiments, the module 128 may determine the “most effective” treatment therapy. In some embodiments, the module 128 may optimize one or more effective therapies, including the most effective, by one or more criteria. For example, the one or more optimization criteria may include but is not limited to constraining the corresponding treatment/treatment combination, doses, and schedule to (i) a maximum cumulative dosage per day for each therapeutic and/or maximum number of administrations (e.g., infusion times) during treatment, (ii) minimum absolute number of neutrophils during treatment, (iii) among others, or any combination thereof. For example, the optimization module 128, may determine a treatment combination, dose, and/or schedule to be inputted into the block 210 and to be used by steps 220-240 to determine one or more metrics. In some embodiments, the optimization module 128 can cause steps 220-240 to be performed using different treatment information until a minimum absolute number of neutrophils are determined in step 240 with a treatment information that is below a maximum cumulative dosage per day (or total for each cycle) for each therapeutic and/or the maximum number of administrations (e.g., infusion times) during treatment.

At block 260, the method 200 may include generating visualizations of the determined metrics for one or more (effective and/or optimized) treatment therapies and/or therapeutic target(s) and/or disease state(s) simulated for a period of time (treatment period/course) from steps 240 and/or 250, for example, by the metric reporting module 130. FIGS. 6A-14, discussed below, show examples of visualizations of the metrics determined using the methods and systems according to embodiments.

At block 270, the treatment may be administered, for example, according to the treatment therapy determined in step 250 and/or determined by a clinician.

At blocks 280 and 290, the method 200 may optionally include a comparison of standard of care laboratory results and simulations. For example, using the patient information 214, using one or more tests, collected at one or more time points after administration of treatment, the system 110, for example, the assessment module 132 may compare the collected results with the metrics determined in step 240 corresponding to the treatment administered. If the module 132 determines at step 280 that the determined metrics have a low accuracy (outside a threshold) based on the comparison, the assessment module 132 may determine one or more patient-specific parameters, such as sub-clones' cell cycle times and therapeutic-specific sensitivity. If the module 132 determines at step 280 that the determined metrics have high accuracy (within a threshold) based on the comparison, the assessment module 132 may indicate that the treatment should be continued to be administered (at block 270) according to the determined treatment therapy.

FIGS. 6A and 6B show examples of visualizations of dynamic prediction of the absolute number of normal cells and abnormal cells in the BM. FIG. 6A shows a visualization based on an example of a patient under 3 cycles of low-dose Ara-C treatment with one resistant sub-clonal population. FIG. 6B shows a visualization of another patient (under 4 cycles of intensive Ara-C and DNR treatment with the sensitive disease only. These visualizations can allow the clinician to be alerted of the timing of BM failure during treatment and BM recovery after treatment. Therefore, the visualizations generated from the methods and systems of the disclosure can provide crucial information to evaluate treatment efficacy and toxicity and the potential/timing of patient recovery.

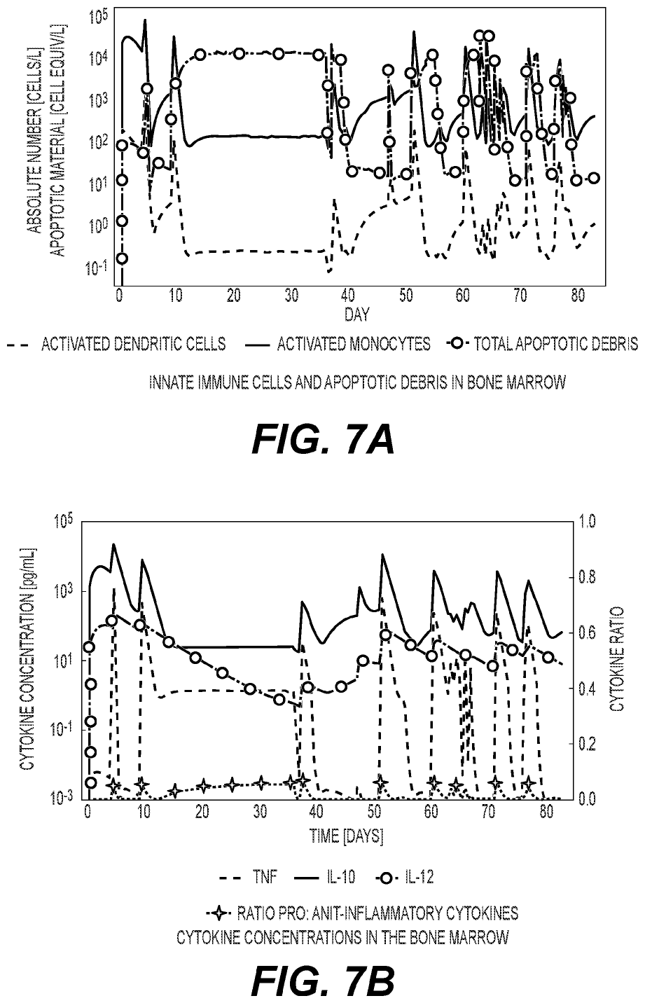

FIGS. 7A and 7B show examples of visualizations of innate immune cells and (i) apoptotic debris and (ii) respective cytokines in the BM, respectively, during treatment. FIG. 7A shows the absolute cell number for activated dendritic cells, activated monocytes, and apoptotic debris that are simulated over the course of treatment. FIG. 7B shows inflammatory TNFα, IL-12 and anti-inflammatory IL-10 cytokines in BM that are simulated over the course of treatment. The visualizations of the dynamic prediction of absolute myeloid-derived innate immune cells and signaling molecules in the BM and PB, such as monocytes, dendritic cells, and macrophages, can allow the clinician to be alerted of the best timing of immune-targeted treatments and to the possibility of severe immune stimulation in response to treatment leading to immune-related adverse events (iRAEs). Therefore, the visualizations generated from the methods and systems of the disclosure can provide critical information for improved responses to immunotherapy, targeted anti-cytokine, cytokine-enhancing and/or cellular therapeutics, and to prevent iRAEs.

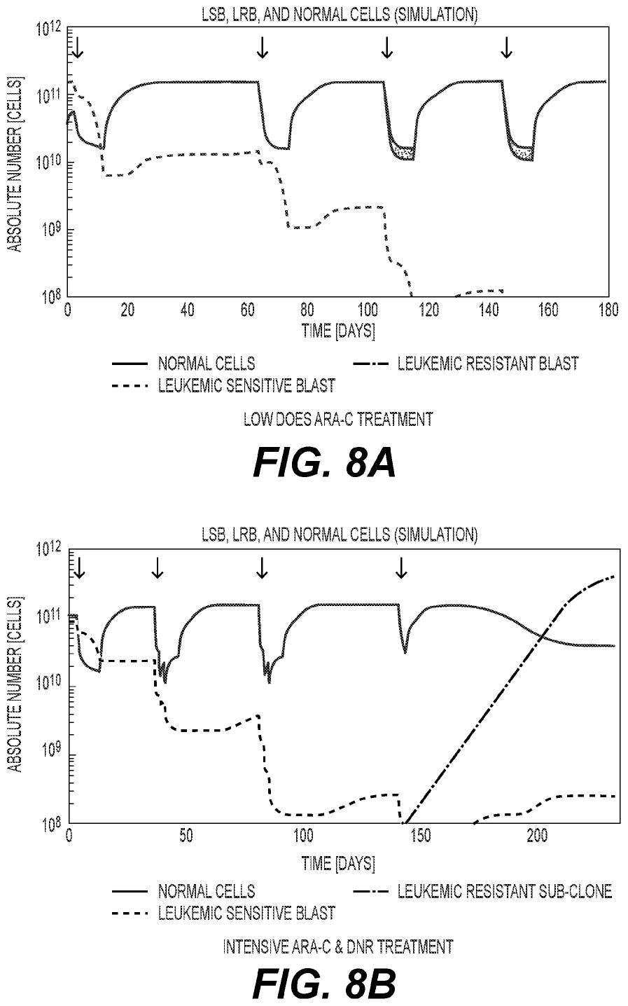

FIGS. 8A and 8B show examples of visualizations of dynamic prediction of total normal cells, and sensitive and resistant sub-clones by showing the absolute number of total normal and abnormal cells in BM in graphs. FIG. 8A shows a visualization based on an example of a patient under 4 cycles of low-dose Ara-C treatment with one sensitive disease clone only. FIG. 8B shows a visualization based on an example of another patient (under 4 cycles of intensive Ara-C and DNR treatment with an initial dominant sensitive sub-clonal population, which is eliminated during treatment and aggressive growth of an initially almost undetectable resistant sub-clone that overtakes the patient bone marrow producing relapse.

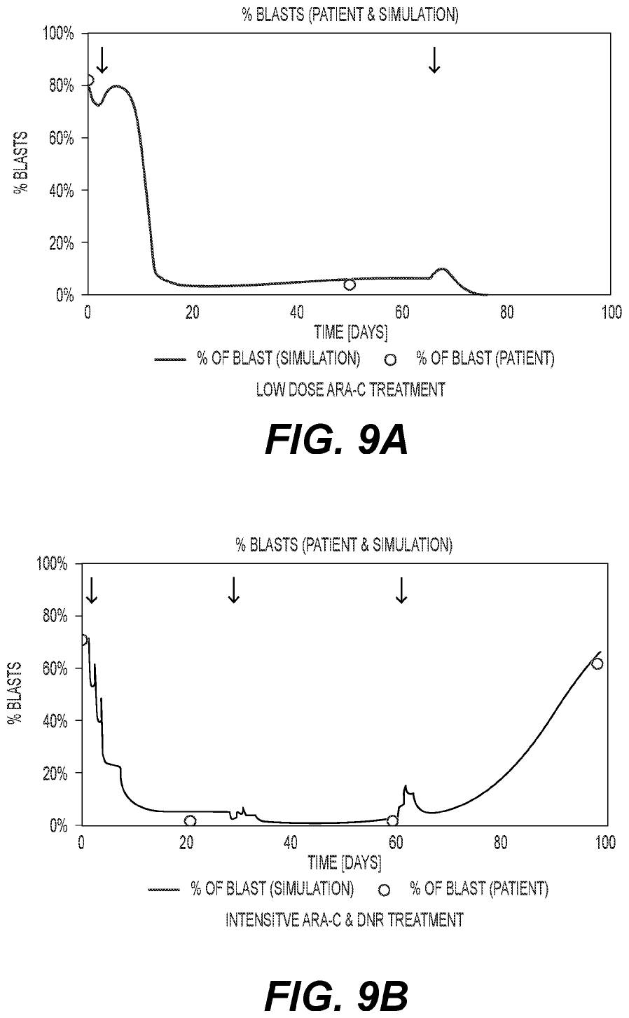

FIGS. 9A and 9B show examples of visualizations of dynamic prediction of the overall percentage of leukemic blasts. By showing the percentage of overall leukemic blasts (addition of all subclones) in graphs, the leukemic burden in BM can be visualized. FIG. 9A shows a visualization based on an example of a patient undergoing treatment with 2 cycles of low-dose Ara-C that achieved complete remission (<5% leukemic blasts). FIG. 9B shows a visualization based on an example of another patient treated with 3 cycles of intensive Ara-C and DNR treatment-the 2 initial cycles were successful, and the 3^{rd} cycle was ineffective due to the predicted growth of a resistant and aggressive sub-clone that overtakes the patient BM causing relapse.

The dynamic prediction of the absolute number of sub-clonal populations (FIGS. 8A and B) and percentage of sub-clones (FIGS. 9A and B) in the BM prior to, during, and after treatment can allow the system to optimize the therapeutic selection, administration route, administration method and regimen schedule based on personalized tumor and normal cell sub-clonal evolutions. Based on this information, the strategic administration of targeted therapies can be recommended in order to maximize effectiveness and to eliminate dominant or aggressive sub-clones for improved disease control and/or cure while avoiding the administration of ineffective therapeutics that can be life-threatening and debilitating, thereby reducing treatment toxicity, and improving outcomes and patient quality of life during and after treatment.

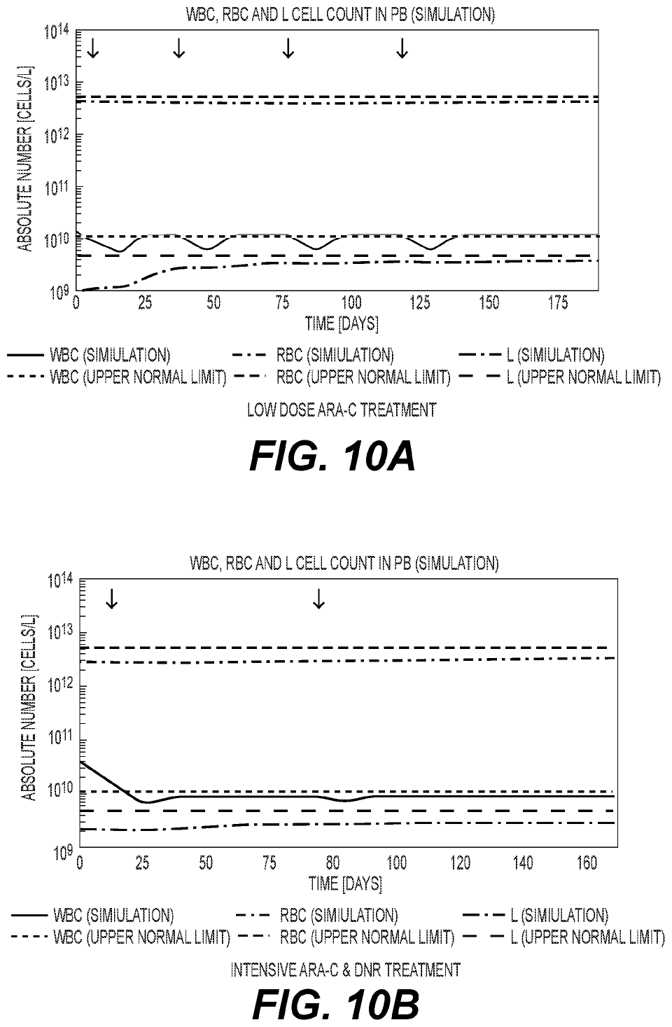

FIGS. 10A and 10B show examples of visualizations of dynamic prediction of peripheral blood recovery by showing the absolute number of RBC, WBC, and L in peripheral blood and the normal level to be achieved for complete hematological recovery (dashed line) in graphs. FIG. 10A shows a visualization based on an example of a patient undergoing 4 cycles of low-dose Ara-C treatment that achieved complete remission and complete hematological recovery. FIG. 10B shows a visualization based on an example of another patient undergoing 2 cycles of intensive Ara-C and DNR that achieved complete remission and is achieving complete hematological recovery.

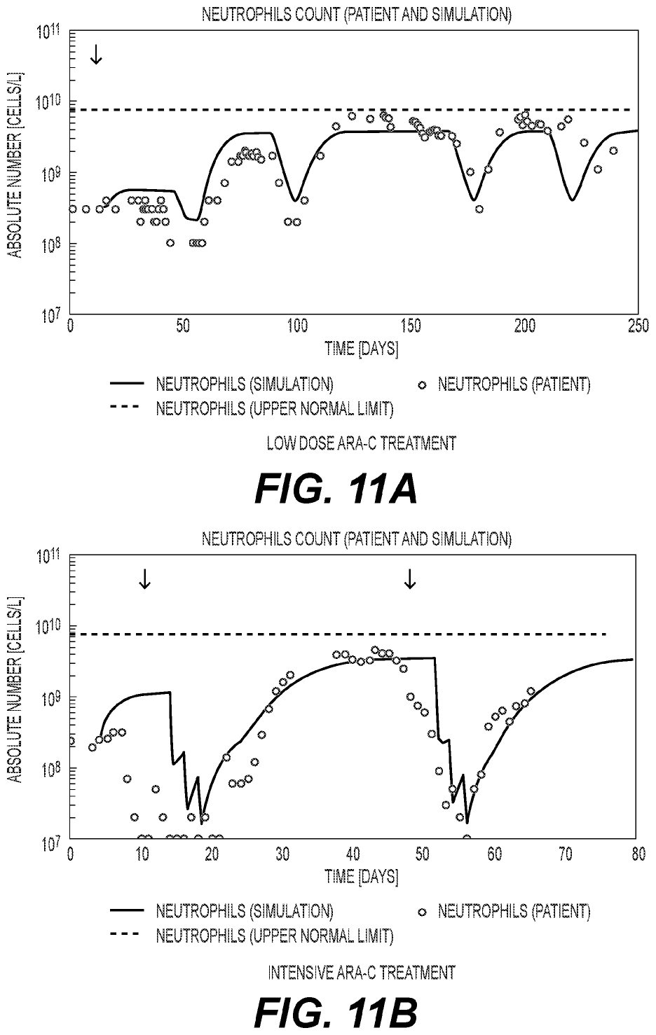

FIGS. 11A and 11B show examples of visualizations of dynamic prediction of absolute neutrophils count (lines) determined using the method according to embodiments, patient daily neutrophil counts during treatment (dots) and the normal level to be achieved for complete hematological recovery (dashed line). FIG. 11A shows a visualization based on an example of a patient undergoing 4 cycles of low-dose Ara-C treatment. The patient initially presented with granulocytic dysplasia that delayed neutrophil recovery. Following treatment, the patient achieved complete remission and complete hematological recovery. FIG. 11B shows a visualization based on an example of another patient undergoing 2 cycles of intensive Ara-C and DNR that achieved complete remission and complete hematological recovery.

The dynamic prediction of the absolute number of mature cells in peripheral blood (FIGS. 10A-11B) can allow the system to alert clinicians about the toxicity risk of life-threatening low blood counts (cytopenias), in particular the risk of neutropenic sepsis, which can be life-threatening if not treated promptly, and predict patient complete hematological recovery.

### Example Outputs of Simulation

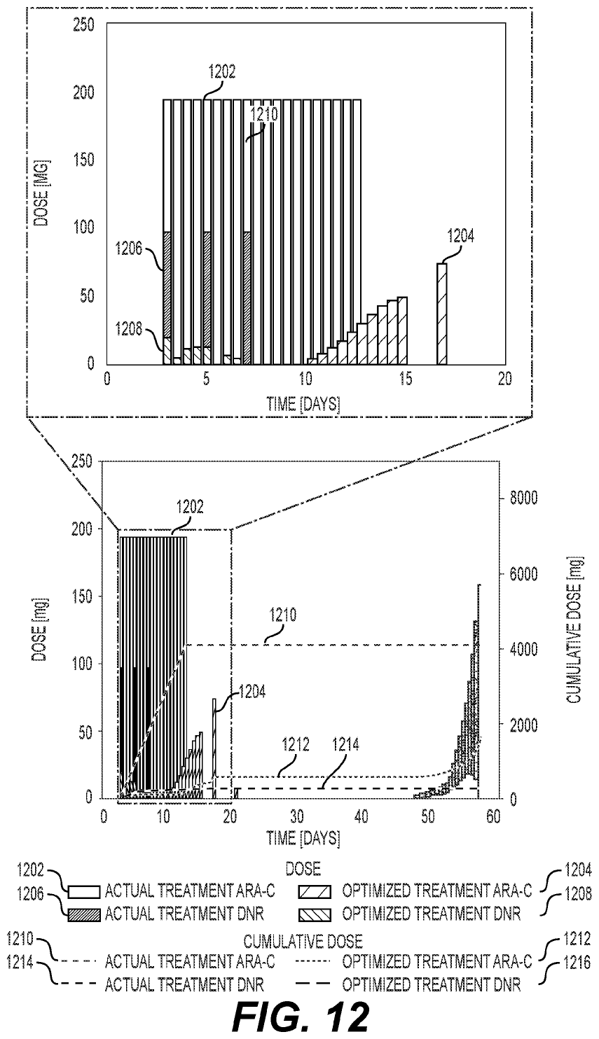

FIGS. 12-14 show examples of results after optimization according to embodiments for one specific patient, which can be outputted in whole or in part in a report, e.g., to be used by a clinician.

Recommended Therapy Profile. FIG. 12 is a diagram showing a comparison of actual treatment received and an (optimized) treatment therapy determined using the systems and methods according to embodiments. FIG. 12, among the other figures, may be an example output report that can be provided to a clinician to guide, based on the exemplary mechanistic simulation, the clinician in altering or adjusting an initial treatment plan for the patient. In FIG. 12, the current treatment plan (shown as “Actual Treatment Data Ara-C”) for the patient is shown as line 1202 along with the simulated cumulative dose (1212) for the current treatment plan, which provides input to the exemplary mechanistic simulation. The initial treatment plan may be based on drug manufacturer-prescribed treatment guidelines. Based on the exemplary mechanistic simulation and optimization operation described herein, the system can present an optimized treatment profile for the patient (shown as “Optimized Treatment Ara-C” and “Optimized Treatment DNR”) per line 1204 along with the cumulative dose data 1212. Similar output is also provided for treatment with DNR (per lines 1206, 1208, 1214, 1216).

As shown through this example illustration, the optimization results can provide personalized, optimal selection of therapeutic, administration route and method, regimen schedule for an individual, or a combination of therapeutics. The optimization can be constrained by comorbidity contraindications, maximum cumulative dose/day or for the treatment course, minimum acceptable blood counts during the treatment, renal or hepatic impairment, risk of myocardial toxicity, and performance status, based on FDA guidance and clinical consortia guidelines/local clinical practice or clinical trial results.

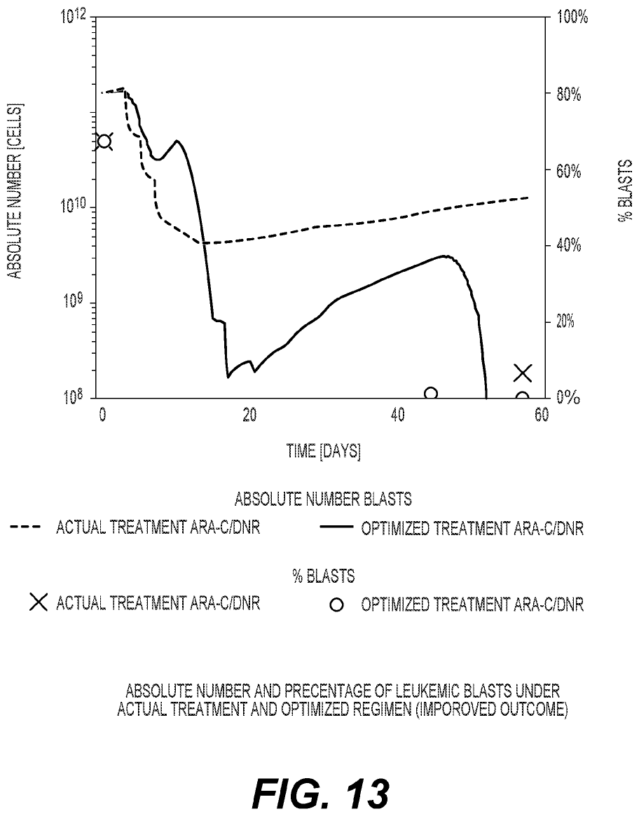

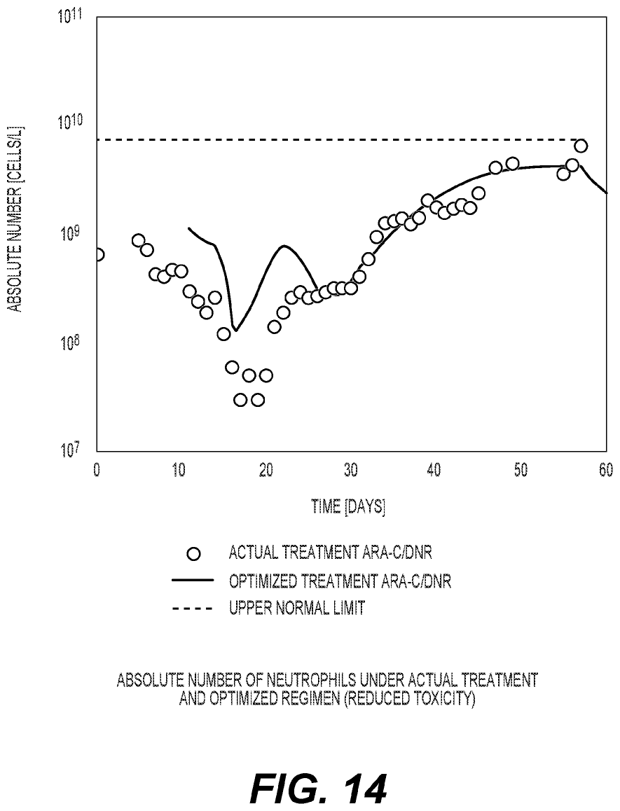

Simulated Patient Improvement. FIG. 13 shows a visual comparison of the absolute number and percentage of leukemic blasts between actual treatment and an (optimized) treatment therapy (regimen) determined using the systems and methods of the disclosure according to embodiments, and FIG. 14 shows a visual comparison of the absolute number of neutrophils between actual treatment (dots) and optimized treatment therapy (regimen) (line) determined using the methods and systems according to embodiments. As shown in FIGS. 13 and 14, the methods and systems of the disclosure can minimize the number of abnormal cells (FIG. 13) to achieve morphologic complete remission, while maintaining the number of normal cells above hematological toxicity levels to reduce the risk of BM failure and ensure recovery, and the absolute number of cells in peripheral blood to avoid cytopenias (FIG. 14).

According to embodiments, the disclosure can intelligently predict tumor evolution so that sequential therapies with therapeutics can be identified for that target: (a) phase of the cell cycle, (b) genomic/transcriptomic expression signatures, (c) metabolomic functional profiles, and (d) immune potential to respond to immunotherapy and/or cell therapy dynamically and to reduce toxic effects of treatment. In this way, patients can have a variety of treatment options that change depending on the predicted kinetics of the heterogenous tumor, which has developed, and the host immune status during treatment. Therefore, the systems and methods of the disclosure can provide personalized treatment simulation and optimization at the bedside in real time.

Example Computing System. FIG. 15 depicts a block diagram of an example computing system 1500 for implementing certain embodiments. For example, in some aspects, the computer system 1500 may include computing systems associated with a device (e.g., the system 110) performing one or more processes (e.g., FIGS. 2-5) disclosed herein. The block diagram illustrates some electronic components or subsystems of the computing system. The computing system 1500 depicted in FIG. 15 is merely an example and is not intended to unduly limit the scope of inventive embodiments recited in the claims. One of ordinary skill in the art would recognize many possible variations, alternatives, and modifications. For example, in some implementations, the computing system 1500 may have more or fewer subsystems than those shown in FIG. 15, may combine two or more subsystems, or may have a different configuration or arrangement of subsystems.

In the example shown in FIG. 15, the computing system 1500 may include one or more processing units 1510 and storage 1520. The processing units 1510 may be configured to execute instructions for performing various operations and can include, for example, a micro-controller, a general-purpose processor, or a microprocessor suitable for implementation within a portable electronic device, such as a Raspberry Pi. The processing units 1510 may be communicatively coupled with a plurality of components within the computing system 1500. For example, the processing units 1510 may communicate with other components across a bus. The bus may be any subsystem adapted to transfer data within the computing system 1500. The bus may include a plurality of computer buses and additional circuitry to transfer data.

In some embodiments, the processing units 1510 may be coupled to the storage 1520. In some embodiments, the storage 1520 may offer both short-term and long-term storage and may be divided into several units. The storage 1520 may be volatile, such as static random access memory (SRAM) and/or dynamic random access memory (DRAM), and/or non-volatile, such as read-only memory (ROM), flash memory, and the like. Furthermore, the storage 1520 may include removable storage devices, such as secure digital (SD) cards. The storage 1520 may provide storage of computer readable instructions, data structures, program modules, audio recordings, image files, video recordings, and other data for the computing system 1500. In some embodiments, the storage 1520 may be distributed into different hardware modules. A set of instructions and/or code might be stored on the storage 1520. The instructions might take the form of executable code that may be executable by the computing system 1500, and/or might take the form of source and/or installable code, which, upon compilation and/or installation on the computing system 1500 (e.g., using any of a variety of generally available compilers, installation programs, compression/decompression utilities, and the like), may take the form of executable code.

In some embodiments, the storage 1520 may store a plurality of application modules 1524, which may include any number of applications, such as applications for controlling input/output (I/O) devices 1540, a switch, a camera, a microphone or audio recorder, a speaker, a media player, a display device, etc.). The application modules 1524 may include particular instructions to be executed by the processing units 1510. In some embodiments, certain applications or parts of the application modules 1524 may be executable by other hardware modules, such as a communication subsystem 1550. In certain embodiments, the storage 1520 may additionally include secure memory, which may include additional security controls to prevent copying or other unauthorized access to secure information.

In some embodiments, the storage 1520 may include an operating system 1522 loaded therein, such as an Android operating system or any other operating system suitable for mobile devices or portable devices. The operating system 1522 may be operable to initiate the execution of the instructions provided by the application modules 1524 and/or manage other hardware modules as well as interfaces with a communication subsystem 1550 which may include one or more wireless or wired transceivers. The operating system 1522 may be adapted to perform other operations across the components of the computing system 1500 including threading, resource management, data storage control, and other similar functionality.

The communication subsystem 1550 may include, for example, an infrared communication device, a wireless communication device and/or chipset (such as a Bluetooth® device, an IEEE 802.11 (Wi-Fi) device, a WiMax device, cellular communication facilities, and the like), NFC, ZigBee, and/or similar communication interfaces. The computing system 1500 may include one or more antennas (not shown in FIG. 15) for wireless communication as part of the communication subsystem 1550 or as a separate component coupled to any portion of the system.

Depending on desired functionality, the communication subsystem 1550 may include separate transceivers to communicate with base transceiver stations and other wireless devices and access points, which may include communicating with different data networks and/or network types, such as wireless wide-area networks (WWANs), WLANs, or wireless personal area networks (WPANs). A WWAN may be, for example, a WiMax (IEEE 802.9) network. A WLAN may be, for example, an IEEE 802.11x network. A WPAN may be, for example, a Bluetooth network, an IEEE 802.15x, or some other types of network. The techniques described herein may also be used for any combination of WWAN, WLAN, and/or WPAN. In some embodiments, the communications subsystem 1550 may include wired communication devices, such as Universal Serial Bus (USB) devices, Universal Asynchronous Receiver/Transmitter (UART) devices, Ethernet devices, and the like. The communications subsystem 1550 may permit data to be exchanged with a network, other computing systems, and/or any other devices described herein. The communication subsystem 1550 may include a means for transmitting or receiving data, such as identifiers of portable goal tracking devices, position data, a geographic map, a heat map, photos, or videos, using antennas and wireless links. The communication subsystem 1550, the processing units 1510, and the storage 1520 may together comprise at least a part of one or more of a means for performing some functions disclosed herein.

The computing system 1500 may include one or more I/O devices 1540, such as sensors, a switch, a camera, a microphone or audio recorder, a communication port, or the like. For example, the I/O devices 1540 may include one or more touch sensors or button sensors associated with the buttons. The touch sensors or button sensors may include, for example, a mechanical switch or a capacitive sensor that can sense the touching or pressing of a button.

In some embodiments, the I/O devices 1540 may include a microphone or audio recorder that may be used to record an audio message. The microphone and audio recorder may include, for example, a condenser or capacitive microphone using silicon diaphragms, a piezoelectric acoustic sensor, or an electret microphone. In some embodiments, the microphone and audio recorder may be a voice-activated device. In some embodiments, the microphone and audio recorder may record an audio clip in a digital format, such as MP3, WAV, WMA, DSS, etc. The recorded audio files may be saved to the storage 1520 or may be sent to the one or more network servers through the communication subsystem 1550.

In some embodiments, the I/O devices 1540 may include a location tracking device, such as a global positioning system (GPS) receiver. In some embodiments, the I/O devices 1540 may include a wired communication port, such as a micro-USB, Lightning, or Thunderbolt transceiver.

The I/O devices 1540 may also include, for example, a speaker, a media player, a display device, a communication port, or the like. For example, the I/O devices 1540 may include a display device, such as an LED or LCD display and the corresponding driver circuit. The I/O devices 1540 may include a text, audio, or video player that may display a text message, play an audio clip, or display a video clip.

The computing system 1500 may include a power device 1560, such as a rechargeable battery for providing electrical power to other circuits on the computing system 1500. The rechargeable battery may include, for example, one or more alkaline batteries, lead-acid batteries, lithium-ion batteries, zinc-carbon batteries, and NiCd or NiMH batteries. The computing system 1500 may also include a battery charger for charging the rechargeable battery. In some embodiments, the battery charger may include a wireless charging antenna that may support, for example, one of Qi, Power Matters Association (PMA), or Association for Wireless Power (A4WP) standard, and may operate at different frequencies. In some embodiments, the battery charger may include a hard-wired connector, such as, for example, a micro-USB or Lightning® connector, for charging the rechargeable battery using a hard-wired connection. The power device 1560 may also include some power management integrated circuits, power regulators, power convertors, and the like.

The computing system 1500 may be implemented in many different ways. In some embodiments, the different components of the computing system 1500 described above may be integrated to a same printed circuit board. In some embodiments, the different components of the computing system 1500 described above may be placed in different physical locations and interconnected by, for example, electrical wires, e.g., over a cloud infrastructure. The computing system 1500 may be implemented in various physical forms and may have various external appearances. The components of computing system 1500 may be positioned based on the specific physical form.

The methods, systems, and devices discussed above are examples. Various embodiments may omit, substitute, or add various procedures or components as appropriate. For instance, in alternative configurations, the methods described may be performed in an order different from that described, and/or various stages may be added, omitted, and/or combined. Also, features described with respect to certain embodiments may be combined in various other embodiments. Different aspects and elements of the embodiments may be combined in a similar manner. Also, technology evolves and, thus, many of the elements are examples that do not limit the scope of the disclosure to those specific examples.

The foregoing method descriptions and the process flow diagrams are provided merely as illustrative examples and are not intended to require or imply that the operations of various embodiments must be performed in the order presented. As will be appreciated by one of skill in the art the order of operations in the foregoing embodiments may be performed in any order. Words such as “thereafter,” “then,” “next,” etc. are not intended to limit the order of the operations; these words are simply used to guide the reader through the description of the methods. Further, any reference to claim elements in the singular, for example, using the articles “a,” “an” or “the” is not to be construed as limiting the element to the singular.

While the terms “first” and “second” are used herein to describe data transmission associated with a subscription and data receiving associated with a different subscription, such identifiers are merely for convenience and are not meant to limit various embodiments to a particular order, sequence, type of network or carrier.

Various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality. Whether such functionality is implemented as hardware or software depends upon the particular application and design constraints imposed on the overall system. Skilled artisans may implement the described functionality in varying ways for each particular application, but such embodiment decisions should not be interpreted as causing a departure from the scope of the claims.

The hardware used to implement various illustrative logics, logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computing systems, (e.g., a combination of a DSP and a microprocessor, a plurality of microprocessors, one or more microprocessors in conjunction with a DSP core, or any other such configuration. Alternatively, some operations or methods may be performed by circuitry that is specific to a given function.

In one or more example embodiments, the functions described may be implemented in hardware, software, firmware, or any combination thereof. If implemented in software, the functions may be stored as one or more instructions or code on a non-transitory computer readable medium or non-transitory processor-readable medium. The operations of a method or algorithm disclosed herein may be embodied in a processor-executable software module, which may reside on a non-transitory computer-readable or processor-readable storage medium. Non-transitory computer-readable or processor-readable storage media may be any storage media that may be accessed by a computer or a processor. By way of example but not limitation, such non-transitory computer-readable or processor-readable media may include RAM, ROM, EEPROM, FLASH memory, CD-ROM or other optical disk storage, magnetic disk storage or other magnetic storage devices, or any other medium that may be used to store desired program code in the form of instructions or data structures and that may be accessed by a computer. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above are also included within the scope of non-transitory computer-readable and processor-readable media. Additionally, the operations of a method or algorithm may reside as one or any combination or set of codes and/or instructions on a non-transitory processor-readable medium and/or computer-readable medium, which may be incorporated into a computer program product.

Those of skill in the art will appreciate that information and signals used to communicate the messages described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Terms, “and” and “or” as used herein, may include a variety of meanings that also is expected to depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics. However, it should be noted that this is merely an illustrative example and claimed subject matter is not limited to this example. Furthermore, the term “at least one of” if used to associate a list, such as A, B, or C, can be interpreted to mean any combination of A, B, and/or C, such as A, AB, AC, BC, AA, ABC, AAB, AABBCCC, and the like.

Further, while certain embodiments have been described using a particular combination of hardware and software, it should be recognized that other combinations of hardware and software are also possible. Certain embodiments may be implemented only in hardware, or only in software, or using combinations thereof. In one example, software may be implemented with a computer program product containing computer program code or instructions executable by one or more processors for performing any or all of the steps, operations, or processes described in this disclosure, where the computer program may be stored on a non-transitory computer readable medium. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation such as by executing computer instructions or code, or processors or cores programmed to execute code or instructions stored on a non-transitory memory medium, or any combination thereof. Processes can communicate using a variety of techniques, including, but not limited to, conventional techniques for inter-process communications, and different pairs of processes may use different techniques, or the same pair of processes may use different techniques at different times.

The disclosures of each and every publication cited herein are hereby incorporated herein by reference in their entirety.

While the disclosure has been described in detail with reference to exemplary embodiments, those skilled in the art will appreciate that various modifications and substitutions may be made thereto without departing from the spirit and scope of the disclosure as set forth in the appended claims. For example, elements and/or features of different exemplary embodiments may be combined with each other and/or substituted for each other within the scope of this disclosure and appended claims.

1. A method for simulating one or more treatment responses to different therapeutics or cell therapy for a patient having a disease to determine one or more metrics for the different therapeutics or therapy, the method comprising:
receiving patient and simulation information for a mechanistic simulation of cell population of a patient, the patient information including a normal cell population information and abnormal cell population information;
performing the mechanistic simulation, including a simulated progression of each cell cycle of abnormal and normal cell populations in and between stages of each cell cycle in a target organ or tissue for a period of time using one or more mechanistic mathematical models and the patient and simulation information to estimate one or more of metrics at intervals during and at an end of the period of time, including one or more disease metrics and/or one or more recovery metrics; and
outputting the one or more of metrics in a report, wherein the output is used to adjust treatment or therapy of the patient for the disease.

2. The method according to claim 1, wherein the target organ or tissue is bone marrow and/or peripheral blood.

3. The method according to claim 1, wherein the one or more disease metrics include at least one of a percentage of diseased cells, a type of diseased cells, minimal/measurable residual disease (MRD), sub-clonal evolution, among others, or a combination thereof.

4. The method according to claim 1, wherein the one or more disease metrics or the one or more recovery metrics include at least one of:
(i) an absolute number of simulated normal stem cells, progenitors, precursors and/or absolute number of mature hematopoietic and/or immune cells,
(ii) an absolute number of simulated mature hematopoietic and/or immune cells in peripheral blood of the patient,
(iii) simulated cytokine or other protein biomarkers identified in the blood or bone marrow target organ or tissue,
(iv) immune-related adverse events, or
(v) a combination thereof.

5. The method according to claim 1, wherein the simulation information includes potential treatment information for one or more potential treatment therapies, wherein the potential treatment information includes pharmacokinetics/pharmacodynamics (PK/PD) parameters specific to each potential treatment therapy of the one or more potential treatment therapies;
the method further comprising:
determining, in the mechanistic simulations, one or more pharmacokinetics or pharmacodynamics (PK/PD) metrics for each of the potential treatment therapies using the pharmacokinetics/pharmacodynamics (PK/PD) parameters of the one or more potential treatment therapies, wherein the one or more pharmacokinetics or pharmacodynamics (PK/PD) metrics are employed in the one or more mechanistic mathematical models to predict the one or more of metrics, including the one or more disease metrics and/or the one or more recovery metrics.

6. The method according to claim 1, wherein the simulation information includes cell therapy information, wherein the mechanistic simulation is performed using the cell therapy information in the one or more mechanistic mathematical models to estimate the one or more disease metrics and/or one or more recovery metrics.

7. The method according to claim 5, wherein the one or more determined disease metrics or one or more recovery metrics include a simulated therapeutic concentration in (i) blood and (ii) the target organ or tissue.

8. The method according to claim 5, wherein the one or more determined disease metrics or one or more recovery metrics include adjustment to cell therapy protocol defined for the patient.

9. The method according to claim 1,
wherein the one or more mechanistic mathematical models include a cell-cycle phase model, and
wherein the performing the mechanistic simulation includes simulating in stages within each phase of each cell cycle using the cell-cycle phase model.

10. The method according to claim 1,
wherein the normal cell population information includes cell type distribution information, and
wherein the abnormal cell population information includes cell type and/or aberrations information, and
wherein the performing the mechanistic simulation employs at least one of (i) the cell type distribution information and (ii) the cell type and/or aberrations information to generate simulation parameters, including at least one of: cell cycle time, simulated amount of cellular metabolism, simulated amount of cell interaction with microenvironment of the target organ, simulated kinetics sensitivity, simulated therapeutic sensitivity, or a combination thereof.

11. The method according to claim 1, wherein the one or more determined disease metrics include sub-clonal kinetics and/or evolution based on the cell type and/or the aberrations of the abnormal cell population.

12. The method according to claim 1, wherein the one or more mechanistic mathematical models for the normal cell population include a hemopoietic differentiation tree data model, wherein the hemopoietic differentiation tree data model is employed to track cell types in the mechanistic simulations.

13. The method according to claim 1, wherein:
each simulated progression of the cell cycle of the normal cell populations in and between stages of each cell cycle employs hematopoietic cell type simulation parameters; and
each simulated progression of the cell cycle of the abnormal cell populations in and between stages of each cell cycle employs hematopoietic cell type and aberration types simulation parameters.

14. The method according to claim 13,
wherein each simulated progression of the cell cycle of the normal cell populations in and between stages of each cell cycle employs cell cycling time, recruitment rate, apoptosis rate, and/or sensitivity to therapeutics simulation parameters; and
wherein each simulated progression of the cell cycle of the abnormal cell populations in and between stages of each cell cycle employs cell cycle time and/or sensitivity to therapeutics simulation parameters.

15. A system comprising:
a processor; and
a memory having instructions stored thereon, wherein execution of the instructions by the processor causes the processor to:
receive patient and simulation information for a mechanistic simulation of cell population of a patient, the patient information including a normal cell population information and abnormal cell population information;
perform the mechanistic simulation, including a simulated progression of each cell cycle of abnormal and normal cell populations in and between stages of each cell cycle in a target organ or tissue for a period of time using one or more mechanistic mathematical models and the patient and simulation information to estimate one or more of metrics at intervals during and at an end of the period of time, including one or more disease metrics and/or one or more recovery metrics; and
output the one or more of metrics in a report, wherein the output is used to adjust treatment or therapy of the patient for the disease.

16. A non-transitory computer readable medium having instructions stored thereon, wherein execution of the instructions by a processor causes the processor to;
receive patient and simulation information for a mechanistic simulation of cell population of a patient, the patient information including a normal cell population information and abnormal cell population information:
perform the mechanistic simulation, including a simulated progression of each cell cycle of abnormal and normal cell populations in and between stages of each cell cycle in a target organ or tissue for a period of time using one or more mechanistic mathematical models and the patient and simulation information to estimate one or more of metrics at intervals during and at an end of the period of time, including one or more disease metrics and/or one or more recovery metrics; and
output the one or more of metrics in a report, wherein the output is used to adjust treatment or therapy of the patient for the disease.

17. The non-transitory computer readable medium of claim 16, wherein the instructions include a mechanistic mathematical model to simulate progression of cell cycles of abnormal and normal cell populations in and between stages of each cell cycle in a target organ or tissue for a period of time to generate one or more disease metrics and/or one or more recovery metrics.

18. (canceled)

19. The system of claim 15, wherein the instructions, when executed by the processor, cause the processor to:
determine, in the mechanistic simulations, one or more pharmacokinetics or pharmacodynamics (PK/PD) metrics for each of the potential treatment therapies using the pharmacokinetics/pharmacodynamics (PK/PD) parameters of the one or more potential treatment therapies, wherein the one or more pharmacokinetics or pharmacodynamics (PK/PD) metrics are employed in the one or more mechanistic mathematical models to predict the one or more of metrics, including the one or more disease metrics and/or the one or more recovery metrics.

20. The system of claim 15, wherein the instructions include a mechanistic mathematical model to simulate progression of cell cycles of abnormal and normal cell populations in and between stages of each cell cycle in a target organ or tissue for a period of time to generate one or more disease metrics and/or one or more recovery metrics.
